SEQUENCE LISTING (2 sequences)

SEQ ID NO: 1 - DNA, 20 bp, Artificial Sequence
  Synthetic Sequence
  27F (1)..(20)
agagtttgatcctggctcag

SEQ ID NO: 2 - DNA, 17 bp, Artificial Sequence
  Synthetic Sequence
  534R (1)..(17)
attaccgcggctgctgg

(19) United States
(12) Patent Application Publication
(10) Pub. No.: US 2021/0130852 A1
(43) Pub. Date: May 6, 2021

(54) DEVICE AND METHOD FOR INCREASING THE ORGANIC YIELD OF A BIOLIQUID

(71) Applicants: Georg Ørnskov Rønsch, Vejen (DK); Sebastian Buch Antonsen, Horsens (DK); Jacob Wagner Jensen, Egtved (DK)

(72) Inventors: Georg Ørnskov Rønsch, Vejen (DK); Sebastian Buch Antonsen, Horsens (DK); Jacob Wagner Jensen, Egtved (DK)

(73) Assignee: Renescience A/S, Fredericia (DK)

(21) Appl. No.: 17/005,798

(22) Filed: Aug. 28, 2020

Related U.S. Application Data
(63) Continuation of application No. 14/407,355, filed on Dec. 11, 2014, now abandoned.
(60) Provisional application No. 61/658,419, filed on Jun. 12, 2012.

(51) Int. Cl. C12P 7/08; C12P 19/02; C12P 19/14; C12P 7/62; B09B 5/00

(52) U.S. Cl. CPC C12P 7/08; C12P 19/02; B09B 5/00; C12P 7/62; C12P 19/14

(57) ABSTRACT

Methods of processing municipal solid wastes (MSW) are provided whereby concurrent enzymatic hydrolysis and microbial fermentation of wastes results in liquefaction of biodegradable components as well as accumulation of microbial metabolites. Liquefied biodegradable components are then separated from nondegradable solids to produce a bioliquid characterized in comprising a large percentage of dissolved solids of which a large fraction comprises some combination of acetate, ethanol, butyrate, lactate, formate or propionate. This bioliquid is, itself, a novel biomethane substrate composition, which permits very rapid conversion to biomethane. Methods of biomethane production are further provided using this bioliquid and using other biomethane substrate compositions produced by concurrent enzymatic hydrolysis and microbial fermentation of organic materials.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 88436_0032_Seq_List_8-28-2020.txt, a creation date of Aug. 28, 2020, and a size of 1 Kb. The sequence listing filed via EFS-Web is part of the specification and is hereby incorporated in its entirety by reference herein.

Municipal solid wastes (MSW), particularly including domestic household wastes, wastes from restaurants and food processing facilities, and wastes from office buildings comprise a very large component of organic material that can be further processed to energy, fuels and other useful products. At present only a small fraction of available MSW is recycled, the great majority being dumped into landfills.

Considerable interest has arisen in development of efficient and environmentally friendly methods of processing solid wastes, to maximize recovery of their inherent energy potential and, also, recovery of recyclable materials. One significant challenge in “waste to energy” processing has been the heterogeneous nature of MSW. Solid wastes typically comprise a considerable component of organic, degradable material intermingled with plastics, glass, metals and other non-degradable materials. Unsorted wastes can be directly used in incineration, as is widely practiced in countries such as Denmark and Sweden, which rely on district heating systems. (Strehlik 2009). However, incineration methods are associated with negative environmental consequences and do not accomplish effective recycling of raw materials. Clean and efficient use of the degradable component of MSW combined with recycling typically requires some method of sorting to separate degradable from non-degradable material.

The degradable component of MSW can be used in “waste to energy” processing using both thermo-chemical and biological methods. MSW can be subject to pyrolysis or other modes of thermo-chemical gasification. Organic wastes thermally decomposed at extreme high temperatures, produce volatile components such as tar and methane as well as a solid residue or “coke” that can be burned with less toxic consequences than those associated with direct incineration. Alternatively, organic wastes can be thermally converted to “syngas,” comprising carbon monoxide, carbon dioxide and hydrogen, which can be further converted to synthetic fuels. See e.g. Malkow 2004 for review.

Biological methods for conversion of degradable components of MSW include fermentation to produce specific useful end products, such as ethanol. See e.g. WO2009/150455; WO2009/095693; WO2007/036795; Ballesteros et al. 2010; Li et al 2007.

Alternatively, biological conversion can be achieved by anaerobic digestion to produce biomethane or “biogas.” See e.g. Hartmann and Ahring 2006 for review. Pre-sorted organic component of MSW can be converted to biomethane directly, see e.g. US2004/0191755, or after a comparatively simple “pulping” process involving mincing in the presence of added water, see e.g. US2008/0020456.

However, pre-sorting of MSW to obtain the organic component is typically costly, inefficient or impractical. Source-sorting requires large infrastructure and operating expenses as well as the active participation and support from the community from which wastes are collected—an activity which has proved difficult to achieve in modern urban societies. Mechanical sorting is typically capital intensive and further associated with a large loss of organic material, on the order of at least 30% and often much higher. See e.g. Connsonni 2005.

Some of these problems with sorting systems have been successfully avoided through use of liquefaction of organic, degradable components in unsorted waste. Liquefied organic material can be readily separated from non-degradable materials. Once liquefied into a pumpable slurry, organic component can be readily used in thermo-chemical or biological conversion processes. Liquefaction of degradable components has been widely reported using high pressure, high temperature “autoclave” processes, see e.g. US2013/0029394; US2012/006089; US20110008865; WO2009/150455; WO2009/108761; WO2008/081028; US2005/0166812; US2004/0041301; U.S. Pat. Nos. 5,427,650; 5,190,226.

A radically different approach to liquefaction of degradable organic components is that this may achieved using biological process, specifically through enzymatic hydrolysis, see Jensen et al. 2010; Jensen et al. 2011; Tonini and Astrup 2012; WO2007/036795; WO2010/032557.

Enzymatic hydrolysis offers unique advantages over “autoclave” methods for liquefaction of degradable organic components. Using enzymatic liquefaction, MSW processing can be conducted in a continuous manner, using comparatively cheap equipment and non-pressurized reactions run at comparatively low temperatures. In contrast, “autoclave” processes must be conducted in batch mode and generally involve much higher capital costs.

A perceived need for “sterilization” so as to reduce possible health risks posed by MSW-bourne pathogenic microogranisms has been a prevailing theme in support of the predominance of “autoclave” liquefaction methods. See e.g. WO2009/150455; WO2000/072987; Li et al. 2012; Ballesteros et al. 2010; Li et al. 2007. Similarly, it was previously believed that enzymatic liquefaction required thermal pre-treatment to a comparatively high temperature of at least 90-95° C. This high temperature was considered essential, in part to effect a “sterilization” of unsorted MSW and also so that degradable organic components could be softened and paper products “pulped.” See Jensen et al. 2010; Jensen et al. 2011; Tonini and Astrup 2012.

We have discovered that safe enzymatic liquefaction of unsorted MSW can be achieved without high temperature pre-treatment. Indeed, contrary to expectations, high temperature pre-treatment is not only unnecessary, but can be actively detrimental, since this kills ambient microorganisms which are thriving in the waste. Promoting microbial fermentation concurrently with enzymatic hydrolysis at thermophillic conditions >45° C. improves “organic capture,” either using “ambient” microorganisms or using selectively “inoculated” organisms. That is, concurrent thermophillic microbial fermentation safely increases the organic yield of “bioliquid,” which is our term for the liquefied degradable components obtained by enzymatic hydrolysis. Under these conditions, pathogenic microogranisms typically found in MSW do not thrive. See e.g. Hartmann and Ahring 2006; Deportes et al. 1998; Carrington et al. 1998; Bendixen et al. 1994; Kubler et al. 1994; Six and De Baerre et al. 1992. Under these conditions, typical MSW-bourne pathogens are easily outcompeted by ubiquitous lactic acid bacteria and other safe organisms.

In addition to improving “organic capture” from enzymatic hydrolysis, concurrent microbial fermentation using any combination of lactic acid bacteria, or acetate-, ethanol-, formate-, butyrate-, lactate-, pentanoate- or hexanoate-producing microorganisms, “pre-conditions” the bioliquid so as to render it more efficient as a substrate for biomethane production. Microbial fermentation produces bioliquid having a generally increased percentage of dissolved compared with suspended solids, relative to bioliquid produced by enzymatic liquefaction alone. Higher chain polysaccharides are generally more thoroughly degraded due to microbial “pre-conditioning.” Concurrent microbial fermentation and enzymatic hydrolysis degrades biopolymers into readily usable substrates and, further, achieves metabolic conversion of primary substrates to short chain carboxylic acids and/or ethanol. The resulting bioliquid comprising a high percentage of microbial metabolites provides a biomethane substrate which effectively avoids the rate limiting “hydrolysis” step, see e.g. Delgenes et al. 2000; Angelidaki et al. 2006; Cysneiros et al. 2011, and which offers further advantages for methane production, particularly using very rapid “fixed filter” anaerobic digestion systems.

### SUMMARY

### Brief Description of the Figures

FIG. 1. Conversion of dry matter expressed as dry matter recovered in supernatant as a percent of total dry matter in concurrent enzymatic hydrolysis and microbial fermentation stimulated by inoculation with EC12B bioliquid from example 5.

FIG. 2. Bacterial metabolites recovered in supernatant following concurrent enzymatic hydrolysis and fermentation induced by addition of bioliquid from example 5.

FIG. 3. Graphical presentation of the REnescience test-reactor.

FIG. 4. Schematic illustration of demonstration plant set-up.

FIG. 5. Organic capture in bioliquid during different time period expressed as kg VS per kg MSW processed.

FIG. 6. Bacterial metabolites expressed as a percent of dissolved VS in bioliquid as well as aerobic bacterial counts at different time points during the experiment.

FIG. 7. Distribution of bacterial species identified in bioliquid from example 3.

FIG. 8 Distribution of the 13 predominant bacteria in the EC12B sampled from the test described in example 5.

FIG. 9. Biomethane production ramp up and ramp down using bioliquid from example 5.

FIG. 10 Biomethane production “ramp up” and “ramp down” characterization of the “high lactate” bioliquid from example 2.

FIG. 11 Biomethane production “ramp up” and “ramp down” characterization of the “low lactate” bioliquid from example 2.

FIG. 12 shows biomethane production “ramp up” characterization of the hydrolysed wheat straw bioliquid.

### DETAILED DESCRIPTION OF EMBODIMENTS

In some embodiments, the invention provides a method of processing municipal solid waste (MSW) comprising the steps of

(i). providing MSW at a non-water content of between 5 and 40% and at a temperature of between 45 and 75 degrees C.,
(ii). enzymatically hydrolysing the biodegradable parts of the MSW concurrently with microbial fermentation at a temperature between 45 and 75 degrees C. resulting in liquefaction of biodegradable parts of the waste and accumulation of microbial metabolites, followed by
(iii). sorting of the liquefied, biodegradble parts of the waste from non-biodegradable solids to produce a bioliquid characterized in comprising dissolved volatile solids of which at least 25% by weight comprise any combination of acetate, butyrate, ethanol, formate, lactate and/or propionate, followed by
(iv). anaerobic digestion of the bioliquid to produce biomethane.

In some embodiments, the invention provides an organic liquid biogas substrate produced by enzymatic hydrolysis and microbial fermentation of municipal solid waste (MSW) characterized in that
at least 40% by weight of the non-water content exists as dissolved volatile solids, which dissolved volatile solids comprise at least 25% by weight of any combination of acetate, butyrate, ethanol, formate, lactate and/or propionate.

In some embodiments, the invention provides a method of producing biogas comprising the steps of

(i). providing an organic liquid biogas substrate pre-conditioned by microbial fermentation such that at least 40% by weight of the non-water content exists as dissolved volatile solids, which dissolved volatile solids comprise at least 25% by weight of any combination of acetate, butyrate, ethanol, formate, lactate and/or propionate,
(ii). transferring the liquid substrate into an anaerobic digestion system, followed by
(iii). conducting anaerobic digestion of the liquid substrate to produce biomethane.

The metabolic dynamics of microbial communities engaged in anaerobic digestion are complex. See Supaphol et al. 2010; Morita and Sasaki 2012; Chandra et al. 2012. In typical anaerobic digestion (AD) for production of methane biogas, biological processes mediated by microorganisms achieve four primary steps—hydrolysis of biological marcomolecules into constituent monomers or other metabolites; acidogenesis, whereby short chain hydrocarbon acids and alcohols are produced; acetogenesis, whereby available nutrients are catabolized to acetic acid, hydrogen and carbon dioxide; and methanogenesis, whereby acetic acid and hydrogen are catabolized by specialized archaea to methane and carbon dioxide. The hydrolysis step is typically rate-limiting See e.g. Delgenes et al. 2000; Angelidaki et al. 2006; Cysneiros et al. 2011.

Accordingly, it is advantageous in preparing substrates for biomethane production that these be previously hydrolysed through some form of pretreatment. In some embodiments, methods of the invention combine microbial fermentation with enzymatic hydrolysis of MSW as both a rapid biological pretreatment for eventual biomethane production as well as a method of sorting degradable organic components from otherwise unsorted MSW.

Biological pretreatments have been reported using solid biomethane substrates including source-sorted organic component of MSW. See e.g. Fdez-Guelfo et al. 2012; Fdez-Guelfo et al. 2011 A; Fdez-Guelfo et al. 2011 B; Ge et al. 2010; Lv et al. 2010; Borghi et al. 1999. Improvements in eventual methane yields from anaerobic digestion were reported as a consequence of increased degradation of complex biopolymers and increased solubilisation of volatile solids. However the level of solubilisation of volatile solids and the level of conversion to volatile fatty acids achieved by these previously reported methods do not even approach the levels achieved by methods of the invention. For example, Fdez-Guelfo et al. 2011 A report a 10-50% relative improvement in solubilisation of volatile solids achieved through various biological pretreatments of pre-sorted organic fraction from MSW—this corresponds to final absolute levels of solubilisation between about 7 to 10% of volatile solids. In contrast, methods of the invention produce liquid biomethane substrates comprising at least 40% dissolved volatile solids.

Two-stage anaerobic digestion systems have also been reported in which the first stage process hydrolyses biomethane substrates including source-sorted organic component of MSW and other specialized biogenic substrates. During the first anaerobic stage, which is typically thermophillic, higher chain polymers are degraded and volatile fatty acids produced. This is followed by a second stage anaerobic stage conducted in a physically separate reactor in which methanogenesis and acetogenesis dominate. Reported two-stage anaerobic digestion systems have typically utilized source-sorted, specialized biogenic substrates having less than 7% total solids. See e.g. Supaphol et al. 2011; Kim et al. 2011; Lv et al. 2010; Riau et al. 2010; Kim et al. 2004; Schmit and Ellis 2000; Lafitte-Trouque and Forster 2000; Dugba and Zhang 1999; Kaiser et al. 1995; Harris and Dague 1993. More recently, some two stage AD systems have been reported which utilize source-sorted, specialized biogenic substrates at levels as high as 10% total solids. See e.g. Yu et al. 2012; Lee et al. 2010; Zhang et al. 2007. Certainly none of the reported two-stage anaerobic digestion systems has ever contemplated use of unsorted MSW as a substrate, much less in order to produce a high solids liquid biomethane substrate. Two stage anaerobic digestion seeks to convert solid substrates, continuously feeding additional solids to and continuously removing volatile fatty acids from the first stage reactor.

Any suitable solid waste may be used to practice methods of the invention. As will be understood by one skilled in the art, the term “municipal solid waste” (MSW) refers to waste fractions which are typically available in a city, but that need not come from any municipality per se. MSW can be any combination of cellulosic, plant, animal, plastic, metal, or glass waste including but not limited to any one or more of the following: Garbage collected in normal municipal collections systems, optionally processed in some central sorting, shredding or pulping device such Dewaster® or reCulture®; solid waste sorted from households, including both organic fractions and paper rich fractions; waste fractions derived from industry such as restaurant industry, food processing industry, general industry; waste fractions from paper industry; waste fractions from recycling facilities; waste fractions from food or feed industry; waste fraction from the medicinal industry; waste fractions derived from agriculture or farming related sectors; waste fractions from processing of sugar or starch rich products; contaminated or in other ways spoiled agriculture products such as grain, potatoes and beets not exploitable for food or feed purposes; garden refuse.

MSW is by nature typically heterogeneous. Statistics concerning composition of waste materials are not widely known that provide firm basis for comparisons between countries. Standards and operating procedures for correct sampling and characterisation remain unstandardized. Indeed, only a few standardised sampling methods have been reported. See e.g. Riber et al., 2007. At least in the case of household waste, composition exhibits seasonal and geographical variation. See e.g. Dahlen et al., 2007; Eurostat, 2008; Hansen et al., 2007b; Muhle et al., 2010; Riber et al., 2009; Simmons et al., 2006; The Danish Environmental Protection agency, 2010. Geographical variation in household waste composition has also been reported, even over small distances of 200-300 km between municipalities (Hansen et al., 2007b).

In some embodiments, MSW is processed as “unsorted” wastes. The term “unsorted” as used herein refers to a process in which MSW is not substantially fractionated into separate fractions such that biogenic material is not substantially separated from plastic and/or other inorganic material. Wastes may be “unsorted” as used herein notwithstanding removal of some large objects or metal objects and notwithstanding some separation of plastic and/or inorganic material. “Unsorted” waste as used herein refers to waste that has not been substantially fractionated so as to provide a biogenic fraction in which less than 15% of the dry weight is non-biogenic material. Waste that comprises a mixture of biogenic and non-biogenic material in which greater than 15% of the dry weight is non-biogenic material is “unsorted” as used herein. Typically unsorted MSW comprises biogenic wastes, meaning wastes which can be degraded to biologically convertible substances, including food and kitchen waste, paper- and/or cardboard-containing materials, food wastes and the like; recyclable materials, including glass, bottles, cans, metals, and certain plastics; other burnable materials, which while not practically recyclable per se may give heat value in the form of refuse derived fuels; as well as inert materials, including ceramics, rocks, and various forms of debris.

In some embodiments, MSW can be processed as “sorted” waste. The term “sorted” as used herein refers to a process in which MSW is substantially fractionated into separate fractions such that biogenic material is substantially separated from plastic and/or other inorganic material. Waste that comprises a mixture of biogenic and non-biogenic material in which less than 15% of the dry weight is non-biogenic material is “sorted” as used herein.

In some embodiments, MSW can be source-separated organic waste comprising predominantly fruit, vegetable and/or animal wastes. A variety of different sorting systems can be used in some embodiments, including source sorting, where households dispose of different waste materials separately. Source sorting systems are currently in place in some municipalities in Austria, Germany, Luxembourg, Sweden, Belgium, the Netherlands, Spain and Denmark. Alternatively industrial sorting systems can be used. Means of mechanical sorting and separation may include any methods known in the art including but not limited to the systems described in US2012/0305688; WO2004/101183; WO2004/101098; WO2001/052993; WO2000/0024531; WO1997/020643; WO1995/0003139; CA2563845; U.S. Pat. No. 5,465,847. In some embodiments, wastes may be lightly sorted yet still produce a waste fraction that is “unsorted” as used herein. In some embodiments, unsorted MSW is used in which greater than 15% of the dry weight is non-biogenic material, or greater than 18%, or greater than 20%, or greater than 21%, or greater than 22%, or greater than 23%, or greater than 24%, or greater than 25%.

In practicing methods of the invention, MSW should be provided at a non-water content of between 10 and 45%, or in some embodiments between 12 and 40%, or between 13 and 35%, or between 14 and 30%, or between 15 and 25%. MSW typically comprises considerable water content. All other solids comprising the MSW are termed “non-water content” as used herein. The level of water content used in practicing methods of the invention relates to several interrelated variables. Methods of the invention produce a liquid biogenic slurry. As will be readily understood by one skilled in the art, the capacity to render solid components into a liquid slurry is increased with increased water content. Effective pulping of paper and cardboard, which comprise a substantial fraction of typical MSW, is typically improved where water content is increased. Further, as is well known in the art, enzyme activities can exhibit diminished activity when hydrolysis is conducted under conditions with low water content. For example, cellulases typically exhibit diminished activity in hydrolysis mixtures that have non-water content higher than about 10%. In the case of cellulases, which degrade paper and cardboard, an effectively linear inverse relationship has been reported between substrate concentration and yield from the enzymatic reaction per gram substrate. See Kristensen et al. 2009. Using commercially available isolated enzyme preparations optimized for lignocellulosic biomass conversion, we have observed in pilot scale studies that non-water content can be as high as 15% without seeing clearly detrimental effects.

In some embodiments, some water content should normally be added to the waste in order to achieve an appropriate non-water content. For example, consider a fraction of unsorted Danish household waste. Table 1, which describes characteristic composition of unsorted MSW reported by Riber et al. (2009), “Chemical composition of material fractions in Danish household waste,” Waste Management 29:1251. Riber et al. characterized the component fractions of household wastes obtained from 2220 homes in Denmark on a single day in 2001. It will be readily understood by one skilled in the art that this reported composition is simply a representative example, useful in explaining methods of the invention. In the example shown in Table 1, without any addition of water content prior to mild heating, the organic, degradable fraction comprising vegetable, paper and animal waste would be expected to have approximately 47% non-water content on average. [(absolute % non-water)/(% wet weight)=(7.15+18.76+4.23)/(31.08+23.18+9.88)=47% non-water content.] Addition of a volume of water corresponding to one weight equivalent of the waste fraction being processed would reduce the non-water content of the waste itself to 29.1% (58.2%/2) while reducing the non-water content of the degradable component to about 23.5% (47%/2). Addition of a volume of water corresponding to two weight equivalents of the waste fraction being processed would reduce the non-water content of the waste itself to 19.4% (58.2%/3) while reducing the non-water content of the degradable component to about 15.7% (47%/3).

TABLE 1
Summarised mass distribution of
waste fractions from Denmark 2001

| | | Part of overall waste |
|---|---|---|
| | | expressed as absolute |
| | Part of overall waste | contribution to total non |
| Waste fraction | quantity % wet weight | water content of 58.2% |
| Vegetable waste (a) | 31.08 | 7.15 |
| Paper waste (b) | 23.18 | 18.76 |
| Animal waste (a) | 9.88 | 4.23 |
| Plastic waste (c) | 9.17 | 8.43 |
| Diapers (a) | 6.59 | 3.59 |
| Non combustibles (d) | 4.05 | 3.45 |
| Metal (e) | 3.26 | 2.9 |
| Glass (f) | 2.91 | 2.71 |
| Other (g) | 9.88 | 6.98 |
| TOTAL | 100.00% | 58.20% |

(a) Pure fraction.
(b) Sum of: newspaper, magazines, advertisements, books, office and clean/dirty paper, paper and carton containers, cardboard, carton with plastic, carton with Al foil, dirty cardboard and kitchen tissues.
(c) Sum of: Soft plastic, plastic bottles, other hard plastic and non-recyclable plastic.
(d) Sum of: Soil, Rocks etc., ash, ceramics, cat litter and other non combustibles.
(e) Sum of: Al containers, al foil, metal-like foil, metal containers and other metal.
(f) Sum of: Clear, green, brown and other glass.
(g) Sum of: The remaining 13 material fractions.

One skilled in the art will readily be able to determine an appropriate quantity of water content, if any, to add to wastes in practicing methods of the invention. Typically as a practical matter, notwithstanding some variability in the composition of MSW being processed, it is convenient to add a relatively constant mass ratio of water, in some embodiments between 0.8 and 1.8 kg water per kg MSW, or between 0.5 and 2.5 kg water per kg MSW, or between 1.0 and 3.0 kg water per kg MSW. As a result, the actual non-water content of the MSW during processing may vary within the appropriate range. Depending on the means being used to achieve enzymatic hydrolysis, the appropriate level of non-water content may vary.

Enzymatic hydrolysis can be achieved using a variety of different means. In some embodiments, enzymatic hydrolysis can be achieved using isolated enzyme preparations. As used herein, the term “isolated enzyme preparation” refers to a preparation comprising enzyme activities that have been extracted, secreted or otherwise obtained from a biological source and optionally partially or extensively purified.

A variety of different enzyme activities may be advantageously used to practice methods of the invention. Considering, for example, the composition of MSW shown in Table 1, it will be readily apparent that paper-containing wastes comprise the greatest single component, by dry weight, of the biogenic material. Accordingly, as will be readily apparent to one skilled in the art, for typical household waste, cellulose-degrading activity will be particularly advantageous. In paper-containing wastes, cellulose has been previously processed and separated from its natural occurrence as a component of lignocellulosic biomass, intermingled with lignin and hemicellulose. Accordingly, paper-containing wastes can be advantageously degraded using a comparatively “simple” cellulase preparation.

“Cellulase activity” refers to enzymatic hydrolysis of 1,4-B-D-glycosidic linkages in cellulose. In isolated cellulase enzyme preparations obtained from bacterial, fungal or other sources, cellulase activity typically comprises a mixture of different enzyme activities, including endoglucanases and exoglucanases (also termed cellobiohydrolases), which respectively catalyse endo- and exo-hydrolysis of 1,4-B-D-glycosidic linkages, along with B-glucosidases, which hydrolyse the oligosaccharide products of exoglucanase hydrolysis to monosaccharides. Complete hydrolysis of insoluble cellulose typically requires a synergistic action between the different activities.

As a practical matter, it can be advantageous in some embodiments to simply use a commercially available isolated cellulase preparation optimized for lignocellulosic biomass conversion, since these are readily available at comparatively low cost. These preparations are certainly suitable for practicing methods of the invention. The term “optimized for lignocellulosic biomass conversion” refers to a product development process in which enzyme mixtures have been selected and modified for the specific purpose of improving hydrolysis yields and/or reducing enzyme consumption in hydrolysis of pretreated lignocellulosic biomass to fermentable sugars.

However, commercial cellulase mixtures optimized for hydrolysis of lignocellulosic biomass typically contain high levels of additional and specialized enzyme activities. For example, we determined the enzyme activities present in commercially available cellulase preparations optimized for lignocellulosic biomass conversion and provided by NOVOZYMES™ under the trademarks CELLIC CTEC2™ and CELLIC CTEC3™ as well as similar preparations provided by GENENCOR™ under the trademark ACCELLERASE 1500™ and found that each of these preparations contained endoxylanase activity over 200 U/g, xylosidase activity at levels over 85 U/g, B-L-arabinofuranosidase activity at levels over 9 U/g, amyloglucosidase activity at levels over 15 U/g, and a-amylase activity at levels over 2 U/g.

Simpler isolated cellulase preparations may also be effectively used to practice methods of the invention. Suitable cellulase preparations may be obtained by methods well known in the art from a variety of microorganisms, including aerobic and anaerobic bacteria, white rot fungi, soft rot fungi and anaerobic fungi. As described in ref. 13, R. Singhania et al., “Advancement and comparative profiles in the production technologies using solid-state and submerged fermentation for microbial cellulases,” Enzyme and Microbial Technology (2010) 46:541-549, which is hereby expressly incorporated by reference in entirety, organisms that produce cellulases typically produce a mixture of different enzymes in appropriate proportions so as to be suitable for hydrolysis of lignocellulosic substrates. Preferred sources of cellulase preparations useful for conversion of lignocellulosic biomass include fungi such as species of Trichoderma, Penicillium, Fusarium, Humicola, Aspergillus and Phanerochaete.

In addition to cellulase activity, some additional enzyme activities which can prove advantageous in practicing methods of the invention include enzymes which act upon food wastes, such as proteases, glucoamylases, endoamylases, proteases, pectin esterases, pectin lyases, and lipases, and enzymes which act upon garden wastes, such as xylanases, and xylosidases. In some embodiments it can be advantageous to include other enzyme activities such as laminarases, ketatinases or laccases.

In some embodiments, a selected microorganism that exhibits extra-cellular cellulase activity may be directly inoculated in performing concurrent enzymatic hydrolysis and microbial fermentation, including but not limited to any one or more of the following thermophillic, cellulytic organisms can be inoculated, alone or in combination with other organisms Paenibacillus barcinonensis, see Asha et al 2012, Clostridium thermocellum, see Blume et al 2013 and Lv and Yu 2013, selected species of Streptomyces, Microbispora, and Paenibacillus, see Eida et al 2012, Clostridium straminisolvens, see Kato et al 2004, species of Firmicutes, Actinobacteria, Proteobacteria and Bacteroidetes, see Maki et al 2012, Clostridium clariflavum, see Sasaki et al 2012, new species of Clostridiales phylogenetically and physiologically related to Clostridium thermocellum and Clostridium straminisolvens, see Shiratori et al 2006, Clostridium clariflavum sp. nov. and Clostridium Caenicola, see Shiratori et al 2009, Geobacillus Thermoleovorans, seeTai et al 2004, Clostridium stercorarium, see Zverlov et al 2010, or any one or more of the thermophillic fungi Sporotrichum thermophile, Scytalidium thermophillum, Clostridium straminisolvens and Thermonospora curvata, Kumar et al. 2008 for review. In some embodiments, organisms exhibiting other useful extra cellular enzymatic activities may be inoculated to contribute to concurrent enzymatic hydrolysis and microbial fermentation, for example, proteolytic and keratinolytic fungi, see Kowalska et al. 2010, or lactic acid bacteria exhibiting extra-cellular lipase activity, see Meyers et al. 1996.

Enzymatic hydrolysis can be conducted by methods well known in the art, using one or more isolated enzyme preparations comprising any one or more of a variety of enzyme preparations including any of those mentioned previously or, alternatively, by inoculating the process MSW with one or more selected organisms capable of affecting the desired enzymatic hydrolysis. In some embodiments, enzymatic hydrolysis can be conducted using an effective amount of one or more isolated enzyme preparations comprising cellulase, B-glucosidase, amylase, and xylanase activities. An amount is an “effective amount” where collectively the enzyme preparation used achieves solubilisation of at least 40% of the dry weight of degradable biogenic material present in MSW within a hydrolysis reaction time of 18 hours under the conditions used. In some embodiments, one or more isolated enzyme preparations is used in which collectively the relative proportions of the various enzyme activities is as follows: A mixture of enzyme activities is used such that 1 FPU cellulase activity is associated with at least 31 CMC U endoglucanase activity and such that 1 FPU cellulase activity is associated with at least at least 7 pNPG U beta glucosidase activity. It will be readily understood by one skilled in the art that CMC U refers to carboxymethycellulose units. One CMC U of activity liberates 1 umol of reducing sugars (expressed as glucose equivalents) in one minute under specific assay conditions of 50° C. and pH 4.8. It will be readily understood by one skilled in the art that pNPG U refers to pNPG units. One pNPG U of activity liberates 1 umol of nitrophenol per minute from para-nitrophenyl-B-D-glucopyranoside at 50° C. and pH 4.8. It will be further readily understood by one skilled in the art that FPU of “filter paper units” provides a measure of cellulase activity. As used herein, FPU refers to filter paper units as determined by the method of Adney, B. and Baker, J., Laboratory Analytical Procedure #006, “Measurement of cellulase activity”, Aug. 12, 1996, the USA National Renewable Energy Laboratory (NREL), which is expressly incorporated by reference herein in entirety.

In practicing embodiments of the invention, it can be advantageous to adjust the temperature of the MSW prior to initiation of enzymatic hydrolysis. As is well known in the art, cellulases and other enzymes typically exhibit an optimal temperature range. While examples of enzymes isolated from extreme thermohillic organisms are certainly known, having optimal temperatures on the order of 60 or even 70 degrees C., enzyme optimal temperature ranges typically fall within the range 35 to 55 degrees. In some embodiments, enzymatic hydrolysis are conducted within the temperature range 30 to 35 degrees C., or 35 to 40 degrees C., or 40 to 45 degrees C., or 45 to 50 degrees C., or 50 to 55 degrees C., or 55 to 60 degrees C., or 60 to 65 degrees C., or 65 to 70 degrees C., or 70 to 75 degrees C. In some embodiments it is advantageous to conduct enzymatic hydrolysis and concurrent microbial fermentation at a temperature of at least 45 degrees C., because this is advantageous in discouraging growth of MSW-bourne pathogens. See e.g. Hartmann and Ahring 2006; Deportes et al. 1998; Carrington et al. 1998; Bendixen et al. 1994; Kubler et al. 1994; Six and De Baerre et al. 1992.

Enzymatic hydrolysis using cellulase activity will typically sacchartify cellulosic material. Accordingly, during enzymatic hydrolysis, solid wastes are both saccharified and liquefied, that is, converted from a solid form into a liquid slurry.

Previously, methods of processing MSW using enzymatic hydrolysis to achieve liquefaction of biogenic components have envisioned a need for heating MSW to a temperature considerably higher than that required for enzymatic hydrolysis, specifically to achieve “sterilization” of the waste, followed by a necessary cooling step, to bring the heated waste back down to a temperature appropriate for enzymatic hydrolysis. In practicing methods of the invention, it is sufficient that MSW be simply brought to a temperature appropriate for enzymatic hydrolysis. In some embodiments it can be advantageous to simply adjust MSW to an appropriate non-water content using heated water, administered in such manner so as to bring the MSW to a temperature appropriate for enzymatic hydrolysis. In some embodiments, MSW is heated, either by adding heated water content, or steam, or by other means of heating, within a reactor vessel. In some embodiments, MSW is heated within a reactor vessel to a temperature greater than 30° C. but less than 85° C., or to a temperature of 84° C. or less, or to a temperature of 80° C. or less, or to a temperature of 75° C. or less, or to a temperature of 70° C. or less, or to a temperature of 65° C. or less, or to a temperature of 60° C. or less, or to a temperature of 59° C. or less, or to a temperature of 58° C. or less, or to a temperature of 57° C. or less, or to a temperature of 56° C. or less, or to a temperature of 55° C. or less, or to a temperature of 54° C. or less, or to a temperature of 53° C. or less, or to a temperature of 52° C. or less, or to a temperature of 51° C. or less, or to a temperature of 50° C. or less, or to a temperature of 49° C. or less, or to a temperature of 48° C. or less, or to a temperature of 47° C. or less, or to a temperature of 46° C. or less, or to a temperature of 45° C. or less. In some embodiments, MSW is heated to a temperature not more than 10° C. above the highest temperature at which enzymatic hydrolysis is conducted.

As used herein MSW is “heated to a temperature” where the average temperature of MSW is increased within a reactor to the temperature. As used herein, the temperature to which MSW is heated is the highest average temperature of MSW achieved within the reactor. In some embodiments, the highest average temperature may not be maintained for the entire period. In some embodiments, the heating reactor may comprise different zones such that heating occurs in stages at different temperatures. In some embodiments, heating may be achieved using the same reactor in which enzymatic hydrolysis is conducted. The object of heating is simply to render the majority of cellulosic wastes and a substantial fraction of the plant wastes in a condition optimal for enzymatic hydrolysis. To be in a condition optimal for enzymatic hydrolysis, wastes should ideally have a temperature and water content appropriate for the enzyme activities used for enzymatic hydrolysis.

In some embodiments, it can be advantageous to agitate during heating so as to achieve evenly heated waste. In some embodiments, agitation can comprise free-fall mixing, such as mixing in a reactor having a chamber that rotates along a substantially horizontal axis or in a mixer having a rotary axis lifting the MSW or in a mixer having horizontal shafts or paddles lifting the MSW. In some embodiments, agitation can comprise shaking, stirring or conveyance through a transport screw conveyor. In some embodiments, agitation continues after MSW has been heated to the desired temperature. In some embodiments, agitation is conducted for between 1 and 5 minutes, or between 5 and 10 minutes, or between 10 and 15 minutes, or between 15 and 20 minutes, or between 20 and 25 minutes, or between 25 and 30 minutes, or between 30 and 35 minutes, or between 35 and 40 minutes, or between 40 and 45 minutes, or between 45 and 50 minutes, or between 50 and 55 minutes, or between 55 and 60 minutes, or between 60 and 120 minutes.

Enzymatic hydrolysis is initiated at that point at which isolated enzyme preparations are added. Alternatively, in the event that isolated enzyme preparations are not added, but instead microorganisms that exhibit desired extracellular enzyme activities are used, enzymatic hydrolysis is initiated at that point which the desired microorganism is added.

In practicing methods of the invention, enzymatic hydrolysis is conducted concurrently with microbial fermentation. Concurrent microbial fermentation can be achieved using a variety of different methods. In some embodiments, microorganisms naturally present in the MSW are simply allowed to thrive in the reaction conditions, where the processed MSW has not previously been heated to a temperature that is sufficient to effect a “sterilization.” Typically, microorganisms present in MSW will include organisms that are adapted to the local environment. The general beneficial effect of concurrent microbial fermentation is comparatively robust, meaning that a very wide variety of different organisms can, individually or collectively, contribute to organic capture through enzymatic hydrolysis of MSW. Without wishing to be bound by theory, we consider that co-fermenting microbes individually have some direct effect on degradation of food wastes that are not necessarily hydrolysed by cellulase enzymes. At the same time, carbohydrate monomers and oligomers released by cellulase hydrolysis, in particular, are readily consumed by virtually any microbial species. This gives a beneficial synergy with cellulase enzymes, possibly through release of product inhibition of the enzyme activities, and also possibly for other reasons that are not immediately apparent. The end products of microbial metabolism in any case are typically appropriate for biomethane substrates. The enrichment of enzymatically hydrolysed MSW in microbial metabolites is, thus, already, in and of itself, an improvement in quality of the resulting biomethane substrate. Lactic acid bacteria in particular are ubiquitous in nature and lactic acid production is typically observed where MSW is enzymatically hydrolysed at non-water content between 10 and 45% within the temperature range 45-50%. At higher temperatures, possibly other species of naturally occurring microorganisms may predominate and other microbial metabolites than lactic acid may become more prevalent.

In some embodiments, microbial fermentation can be accomplished by a direct inoculation using one or more microbial species. It will be readily understood by one skilled in the art that one or more bacterial species used for inoculation so as to provide simultaneous enzymatic hydrolysis and fermentation of MSW can be advantageously selected where the bacterial species is able to thrive at a temperature at or near the optimum for the enzymatic activities used.

Inoculation of the hydrolysis mixture so as to induce microbial fermentation can be accomplished by a variety of different means.

In some embodiments, it can be advantageous to inoculate the MSW either before, after or concurrently with the addition of enzymatic activities or with the addition of microorganisms that exhibit extra-cellular cellulase activity. In some embodiments, it can be advantageous to inoculate using one or more species of LAB including but not limited to any one or more of the following, or genetically modified variants thereof: Lactobacillus plantarum, Streptococcus lactis, Lactobacillus casei, Lactobacillus lactis, Lactobacillus curvatus, Lactobacillus sake, Lactobacillus helveticus, Lactobacillus jugurti, Lactobacillus fermentum, Lactobacillus carnis, Lactobacillus piscicola, Lactobacillus coryniformis, Lactobacillus rhamnosus, Lactobacillus maltaromicus, Lactobacillus pseudoplantarum, Lactobacillus agilis, Lactobacillus bavaricus, Lactobacillus alimentarius, Lactobacillus uamanashiensis, Lactobacillus amylophilus, Lactobacillus farciminis, Lactobacillus sharpeae, Lactobacillus divergens, Lactobacillus alactosus, Lactobacillus paracasei, Lactobacillus homohiochii, Lactobacillus sanfrancisco, Lactobacillus fructivorans, Lactobacillus brevis, Lactobacillus ponti, Lactobacillus reuteri, Lactobacillus buchneri, Lactobacillus viridescens, Lactobacillus confusus, Lactobacillus minor, Lactobacillus kandleri, Lactobacillus halotolerans, Lactobacillus hilgardi, Lactobacillus kefir, Lactobacillus collinoides, Lactobacillus vaccinostericus, Lactobacillus delbrueckii, Lactobacillus bulgaricus, Lactobacillus leichmanni, Lactobacillus acidophilus, Lactobacillus salivarius, Lactobacillus salicinus, Lactobacillus gasseri, Lactobacillus suebicus, actobacillus oris, Lactobacillus brevis, Lactobacillus vaginalis, Lactobacillus pentosus, Lactobacillus panis, Lactococcus cremoris, Lactococcus dextranicum, Lactococcus garvieae, Lactococcus hordniae, Lactococcus raffinolactis, Streptococcus diacetylactis, Leuconostoc mesenteroides, Leuconostoc dextranicum, Leuconostoc cremoris, Leuconostoc oenos, Leuconostoc paramesenteroides, Leuconostoc pseudoesenteroides, Leuconostoc citreum, Leuconostoc gelidum, Leuconostoc carnosum, Pediococcus damnosus, Pediococcus acidilactici, Pediococcus cervisiae, Pediococcus parvulus, Pediococcus halophilus, Pediococcus pentosaceus, Pediococcus intermedius, Bifidobacterium longum, Streptococcus thermophilus, Oenococcus oeni, Bifidobacterium breve, and Propionibacterium freudenreichii, or with some subsequently discovered species of LAB or with other species from the genera Enterococcus, Lactobacillus, Lactococcus, Leuconostoc, Pediococcus, or Carnobacterium that exhibit useful capacity for metabolic processes that produce lactic acid.

It will be readily understood by one skilled in the art that a bacterial preparation used for inoculation may comprise a community of different organisms. In some embodiments, naturally occurring bacteria which exist in any given geographic region and which are adapted to thrive in MSW from that region, can be used. As is well known in the art, LAB are ubiquitous and will typically comprise a major component of any naturally occurring bacterial community within MSW.

In some embodiments, MSW can be inoculated with naturally occurring bacteria, by continued recycling of wash waters or process solutions used to recover residual organic material from non-degradable solids. As the wash waters or process solutions are recycled, they gradually acquire higher microbe levels. In some embodiments, microbial fermentation has a pH lowering effect, especially where metabolites comprise short chain carboxylic acids/fatty acids such as formate, acetate, butyrate, proprionate, or lactate. Accordingly in some embodiments it can be advantageous to monitor and adjust pH of the concurrent enzymatic hydrolysis and microbial fermentation mixture. Where wash waters or process solutions are used to increase water content of incoming MSW prior to enzymatic hydrolysis, inoculation is advantageously made prior to addition of enzyme activities, either as isolated enzyme preparations or as microorganisms exhibiting extra-cellular cellulase activity. In some embodiments, naturally occurring bacteria adapted to thrive on MSW from a particular region can be cultured on MSW or on liquefied organic component obtained by enzymatic hydrolysis of MSW. In some embodiments, cultured naturally occurring bacteria can then be added as an inoculum, either separately or supplemental to inoculation using recycled wash waters or process solutions. In some embodiments, bacterial preparations can be added before or concurrently with addition of isolated enzyme preparations, or after some initial period of pre-hydrolysis.

In some embodiments, specific strains can be cultured for inoculation, including strains that have been specially modified or “trained” to thrive under enzymatic hydrolysis reaction conditions and/or to emphasize or de-emphasize particular metabolic processes. In some embodiments, it can be advantageous to inoculate MSW using bacterial strains which have been identified as capable of surviving on phthalates as sole carbon source. Such strains include but are not limited to any one or more of the following, or genetically modified variants thereof: Chryseomicrobium intechense MW 10T, Lysinibaccillus fusiformis NBRC 157175, Tropicibacter phthalicus, Gordonia JDC-2, Arthrbobacter JDC-32, Bacillus subtilis 3C3, Comamonas testosteronii, Comamonas sp E6, Delftia tsuruhatensis, Rhodoccoccus jostii, Burkholderia cepacia, Mycobacterium vanbaalenii, Arthobacter keyseri, Bacillus sb 007, Arthobacter sp. PNPX-4-2, Gordonia namibiensis, Rhodococcus phenolicus, Pseudomonas sp. PGB2, Pseudomonas sp. Q3, Pseudomonas sp. 1131, Pseudomonas sp. CAT1-8, Pseudomonas sp. Nitroreducens, Arthobacter sp AD38, Gordonia sp CNJ863, Gordonia rubripertinctus, Arthobacter oxydans, Acinetobacter genomosp, and Acinetobacter calcoaceticus. See e.g. Fukuhura et al 2012; Iwaki et al. 2012A; Iwaki et al. 2012B; Latorre et al. 2012; Liang et al. 2010; Liang et al. 2008; Navacharoen et al. 2011; Park et al. 2009; Wu et al. 2010; Wu et al. 2011. Phthalates, which are used as plasticizers in many commercial poly vinyl chloride preparations, are leachable and, in our experience, are often present in liquefied organic component at levels that are undesirable. In some embodiments, strains can be advantageously used which have been genetically modified by methods well known in the art, so as to emphasize metabolic processes and/or de-emphasize other metabolic processes including but not limited to processes that consume glucose, xylose or arabinose.

In some embodiments, it can be advantageous to inoculate MSW using bacterial strains which have been identified as capable of degrading lignin. Such strains include but are not limited to any one or more of the following, or genetically modified variants thereof: Comamonas sp B-9, Citrobacter freundii, Citrobacter sp FJ581023, Pandorea norimbergensis, Amycolatopsis sp ATCC 39116, Streptomyces viridosporous, Rhodococcus jostii, and Sphingobium sp. SYK-6. See e.g. Bandounas et al. 2011; Bugg et al. 2011; Chandra et al. 2011; Chen et al. 2012; Davis et al. 2012. In our experience, MSW typically comprises considerable lignin content, which is typically recovered as undigested residual after AD.

In some embodiments, it can be advantageous to inoculate MSW using an acetate-producing bacterial strain, including but not limited to any one or more of the following, or genetically modified variants thereof: Acetitomaculum ruminis, Anaerostipes caccae, Acetoanaerobium noterae, Acetobacterium carbinolicum, Acetobacterium wieringae, Acetobacterium woodii, Acetogenium kivui, Acidaminococcus fermentans, Anaerovibrio lipolytica, Bacteroides coprosuis, Bacteroides propionicifaciens, Bacteroides cellulosolvens, Bacteroides xylanolyticus, Bifidobacterium catenulatum, Bifidobacterium bifidum, Bifidobacterium adolescentis, Bifidobacterium angulatum, Bifidobacterium breve, Bifidobacterium gallicum, Bifidobacterium infantis, Bifidobacterium longum, Bifidobacterium pseudolongum, Butyrivibrio fibrisolvens, Clostridium aceticum, Clostridium acetobutylicum, Clostridium acidurici, Clostridium bifermentans, Clostridium botulinum, Clostridium butyricium, Clostridium cellobioparum, Clostridium formicaceticum, Clostridium histolyticum, Clostridium lochheadii, Clostridium methylpentosum, Clostridium pasteurianum, Clostridium perfringens, Clostridium propionicum, Clostridium putrefaciens, Clostridium sporogenes, Clostridium tetani, Clostridium tetanomorphum, Clostridium thermocellum, Desulfotomaculum orientis, Enterobacter aerogenes, Escherichia coli, Eubacterium limosum, Eubacterium ruminantium, Fibrobacter succinogenes, Lachnospira multiparus, Megasphaera elsdenii, Moorella thermoacetica, Pelobacter acetylenicus, Pelobacter acidigallici, Pelobacter massiliensis, Prevotella ruminocola, Propionibacterium freudenreichii, Ruminococcus flavefaciens, Ruminobacter amylophilus, Ruminococcus albus, Ruminococcus bromii, Ruminococcus champanellensis, Selenomonas ruminantium, Sporomusa paucivorans, Succinimonas amylolytica, Succinivibrio dextrinosolven, Syntrophomonas wolfei, Syntrophus aciditrophicus, Syntrophus gentianae, Treponema bryantii and Treponema primitia.

In some embodiments, it can be advantageous to inoculate MSW using a butyrate-producing bacterial strain, including but not limited to any one or more of the following, or genetically modified variants thereof: Acidaminococcus fermentans, Anaerostipes caccae, Bifidobacterium adolescentis, Butyrivibrio crossotus, Butyrivibrio fibrisolvens, Butyrivibrio hungatei, Clostridium acetobutylicum, Clostridium aurantibutyricum, Clostridium beijerinckii, Clostridium butyricium, Clostridium cellobioparum, Clostridium difficile, Clostridium innocuum, Clostridium kluyveri, Clostridium pasteurianum, Clostridium perfringens, Clostridium proteoclasticum, Clostridium sporosphaeroides, Clostridium symbiosum, Clostridium tertium, Clostridium tyrobutyricum, Coprococcus eutactus, Coprococcus comes, Escherichia coli, Eubacterium barkeri, Eubacterium biforme, Eubacterium cellulosolvens, Eubacterium cylindroides, Eubacterium dolichum, Eubacterium hadrum, Eubacterium halii, Eubacterium limosum, Eubacterium moniliforme, Eubacterium oxidoreducens, Eubacterium ramulus, Eubacterium rectale, Eubacterium saburreum, Eubacterium tortuosum, Eubacterium ventriosum, Faecalibacterium prausnitzii, Fusobacterium prausnitzii, Peptostreptoccoccus vaginalis, Peptostreptoccoccus tetradius, Pseudobutyrivibrio ruminis, Pseudobutyrivibrio xylanivorans, Roseburia cecicola, Roseburia intestinalis, Roseburia hominis and Ruminococcus bromii.

In some embodiments, it can be advantageous to inoculate MSW using a propionate-producing bacterial strain, including but not limited to any one or more of the following, or genetically modified variants thereof: Anaerovibrio lipolytica, Bacteroides coprosuis, Bacteroides propionicifaciens, Bifidobacterium adolescentis, Clostridium acetobutylicum, Clostridium butyricium, Clostridium methylpentosum, Clostridium pasteurianum, Clostridium perfringens, Clostridium propionicum, Escherichia coli, Fusobacterium nucleatum, Megasphaera elsdenii, Prevotella ruminocola, Propionibacterium freudenreichii, Ruminococcus bromii, Ruminococcus champanellensis, Selenomonas ruminantium and Syntrophomonas wolfei.

In some embodiments, it can be advantageous to inoculate MSW using an ethanol-producing bacterial strain, including but not limited to any one or more of the following, or genetically modified variants thereof: Acetobacterium carbinolicum, Acetobacterium wieringae, Acetobacterium woodii, Bacteroides cellulosolvens, Bacteroides xylanolyticus, Clostridium acetobutylicum, Clostridium beijerinckii, Clostridium butyricium, Clostridium cellobioparum, Clostridium lochheadii, Clostridium pasteurianum, Clostridium perfringens, Clostridium thermocellum, Clostridium thermohydrosulfuricum, Clostridium thermosaccharolyticum, Enterobacter aerogenes, Escherichia coli, Klebsiella oxytoca, Klebsiella pneumonia, Lachnospira multiparus, Lactobacillus brevis, Leuconostoc mesenteroides, Paenibacillus macerans, Pelobacter acetylenicus, Ruminococcus albus, Thermoanaerobacter mathranii, Treponema bryantii and Zymomonas mobilis.

In some embodiments, a consortium of different microbes, optionally including different species of bacteria and/or fungi, may be used to accomplish concurrent microbial fermentation. In some embodiments, suitable microorganisms may be selected so as to provide a desired metabolic outcome at the intended reaction conditions, and then inoculated at a high dose level so as to outcompete naturally occurring strains. For example, in some embodiments, it can be advantageous to inoculate using a homofermentive lactate producer, since this provides a higher eventual methane potential in a resulting biomethane substrate than can be provided by a heterofermentive lactate producer.

In some embodiments, enzymatic hydrolysis and concurrent microbial fermentation are conducted using a hydrolysis reactor that provides agitation by free-fall mixing as described in WO2006/056838, and in WO2011/032557.

Following some period of enzymatic hydrolysis and concurrent microbial fermentation, MSW provided at a non-water content between 10 and 45% is transformed such that biogenic or “fermentable” components become liquefied and microbial metabolites accumulate in the aqueous phase. After some period of enzymatic hydrolysis and concurrent microbial fermentation, the liquefied, fermentable parts of the waste are separated from non-fermentable solids. The liquefied material, once separated from non-fermentable solids, is what we term a “bioliquid.” In some embodiments, at least 40% of the non-water content of this bioliquid comprises dissolved volatile solids, or at least 35%, or at least 30%, or at least 25%. In some embodiments, at least 25% by weight of the dissolved volatile solids in the bioliquid comprise any combination of acetate, butyrate, ethanol, formate, lactate, and/or propionate. In some embodiments, at least 70% by weight of the dissolved volatile solids comprises lactate, or at least 60%, or at least 50%, or at least 40%, or at least 30%, or at least 25%.

In some embodiments, separation of non-fermentable solids from liquefied, fermentable parts of the MSW so as to produce a bioliquid characterized in comprising dissolved volatile solids of which at least 25% by weight comprise any combination of acetate, butyrate, ethanol, formate, lactate and/or propionate is conducted in less than 16 hours after the initiation of enzymatic hydrolysis, or in less than 18 hours, or in less than 20 hours, or in less than 22 hours, or in less than 24 hours, or in less than 30 hours, or in less than 34 hours, or in less than 36 hours.

Separation of liquefied, fermentable parts of the waste from non-fermentable solids can be achieved by a variety of means. In some embodiments, this may be achieved using any combination of at least two different separation operations, including but not limited to screw press operations, ballistic separator operations, vibrating sieve operations, or other separation operations known in the art. In some embodiments, the non-fermentable solids separated from fermentable parts of the waste comprise at least about 20% of the dry weight of the MSW, or at least 25%, or at least 30%. In some embodiments, the non-fermentable solids separated from fermentable parts of the waste comprise at least 20% by dry weight of recyclable materials, or at least 25%, or at least 30%, or at least 35%. In some embodiments, separation using at least two separation operations produces a bioliquid that comprises at least 0.15 kg volatile solids per kg MSW processed, or at least 010. It will be readily understood by one skilled in the art that the inherent biogenic composition of MSW is variable. Nevertheless, the figure 0.15 kg volatile solids per kg MSW processed reflects a total capture of biogenic material in typical unsorted MSW of at least 80%. The calculation of kg volatile solids captured in the bioliquid per kg MSW processed can be estimated over a time period in which total yields and total MSW processed are determined.

In some embodiments, after separation of non-fermentable solids from liquefied, fermentable parts of the MSW to produce a bioliquid, the bioliquid may be subject to post-fermentation under different conditions, including different temperature or pH.

The term “dissolved volatile solids” as used here refers to a simple measurement calculated as follows: A sample of bioliquid is centrifuged at 6900 g for 10 minutes in a 50 ml Falcon tube to produce a pellet and a supernatant. The supernatant is decanted and the wet weight of the pellet expressed as a percentage fraction of the total initial weight of the liquid sample. A sample of supernatant is dried at 60 degrees for 48 hours to determine dry matter content. The volatile solids content of the supernatant sample is determined by subtracting from the dry matter measurement the ash remaining after furnace burning at 550° C. and expressed as a mass percentage as dissolved volatile solids in %. An independent measure of dissolved volatile solids is determined by calculation based on the volatile solids content of the pellet. The wet weight fraction of the pellet is applied as a fractional estimate of undissolved solids volume proportion of total intial volume. The dry matter content of the pellet is determined by drying at 60 degrees C. for 48 hours. The volatile solids content of the pellet is determined by subtracting from the dry matter measurement the ash remaining after furnace burning at 550° C. The volatile solids content of the pellet is corrected by the estimated contribution from supernatant liquid given by (1-wet fraction pellet)×(measured supernatant volatile solid %). From the total volatile solids % measured in the original liquid samples is subtracted the (corrected volatile solids % of the pellet)×(fractional estimate of undissolved solids volume proportion of total initial volume) to give an independent estimate of dissolved volatile solids as %. The higher of the two estimates is used in order not to overestimate the percentage of dissolved volatile solids represented by bacterial metabolites.

In some embodiments the invention provides compositions and methods for biomethane production. The preceding detailed discussion concerning embodiments of methods of processing MSW may optionally be applied to embodiments providing methods and compositions for biomethane production. In some embodiments, the method of producing biomethane comprises the steps of

(i). providing an organic liquid biomethane substrate pre-conditioned by microbial fermentation such that at least 40% by weight of the non-water content exists as dissolved volatile solids, which dissolved volatile solids comprise at least 25% by weight of any combination of acetate, butyrate, ethanol, formate, lactate and/or propionate,
(ii). transferring the liquid substrate into an anaerobic digestion system, followed by
(iii). conducting anaerobic digestion of the liquid substrate to produce biomethane.

In some embodiments, the invention provides an organic liquid biomethane substrate produced by enzymatic hydrolysis and microbial fermentation of municipal solid waste (MSW), or of pretreated lignocellusic biomass, alternatively, comprising enzymatically hydrolysed and microbially fermented MSW, or comprising enzymatically hydrolysed and microbially fermented pretreated lignocellulosic biomass characterized in that
at least 40% by weight of the non-water content exists as dissolved volatile solids, which dissolved volatile solids comprise at least 25% by weight of any combination of acetate, butyrate, ethanol, formate, lactate and/or propionate.

As used herein the term “anaerobic digestion system” refers to a fermentation system comprising one or more reactors operated under controlled aeration conditions in which methane gas is produced in each of the reactors comprising the system. Methane gas is produced to the extent that the concentration of metabolically generated dissolved methane in the aqueous phase of the fermentation mixture within the “anaerobic digestion system” is saturating at the conditions used and methane gas is emitted from the system.

In some embodiments, the “anaerobic digestion system” is a fixed filter system. A “fixed filter anaerobic digestion system” refers to a system in which an anaerobic digestion consortium is immobilized, optionally within a biofilm, on a physical support matrix.

In some embodiments, the liquid biomethane substrate comprises at least 8% total solids, or at least 9% total solids, or at least 10% total solids, or at least 11% total solids, or at least 12% total solids, or at least 13% total solids. “Total solids” as used herein refers to both soluble and insoluble solids, and effectively means “non-water content.” Total solids are measured by drying at 60° C. until constant weight is achieved.

In some embodiments, microbial fermentation is conducted under conditions that discourage methane production by methanogens, for example, at pH less than 6.0, or at pH less than 5.8, or at pH less than 5.6, or at pH less than 5.5. In some embodiments, the liquid biomethane substrate comprises less than saturating concentations of dissolved methane. In some embodiments, the liquid biomethane substrate comprises less than 15 mg/L dissolved methane, or less than 10 mg/L, or less than 5 mg/L.

In some embodiments, prior to anaerobic digestion to produce biomethane, one or more components of the dissolved volatile solids may be removed from the liquid biomethane substrate by distillation, filtration, electrodialysis, specific binding, precipitation or other means well known in the art. In some embodiments, ethanol or lactate may be removed from the liquid biomethane substrate prior to anaerobic digestion to produce biomethane.

In some embodiments, a solid substrate such as MSW or fiber fraction from pretreated lignocellulosic biomass, is subject to enzymatic hydrolysis concurrently with microbial fermentation so as to produce a liquid biomethane substrate pre-conditioned by microbial fermentation such that at least 40% by weight of the non-water content exists as dissolved volatile solids, which dissolved volatile solids comprise at least 25% by weight of any combination of acetate, butyrate, ethanol, formate, lactate and/or propionate. In some embodiments, a liquid biomethane substrate having the above mentioned properties is produced by concurrent enzymatic hydrolysis and microbial fermentation of liquefied organic material obtained from unsorted MSW by an autoclave process. In some embodiments, pretreated lignocellulosic biomass can be mixed with enzymatically hydrolysed and microbially fermented MSW, optionally in such manner that enzymatic activity from the MSW-derived biolioquid provides enzymatic activity for hydrolysis of the lignocellulosic substrate to produce a composite liquid biomethane substrate derived from both MSW and pretreated lignocellulosic biomass.

“Soft lignocellulosic biomass” refers to plant biomass other than wood comprising cellulose, hemicellulose and lignin. Any suitable soft lignocellulosic biomass may be used, including biomasses such as at least wheat straw, corn stover, corn cobs, empty fruit bunches, rice straw, oat straw, barley straw, canola straw, rye straw, sorghum, sweet sorghum, soybean stover, switch grass, Bermuda grass and other grasses, bagasse, beet pulp, corn fiber, or any combinations thereof. Lignocellulosic biomass may comprise other lignocellulosic materials such as paper, newsprint, cardboard, or other municipal or office wastes. Lignocellulosic biomass may be used as a mixture of materials originating from different feedstocks, may be fresh, partially dried, fully dried or any combination thereof. In some embodiments, methods of the invention are practiced using at least about 10 kg biomass feedstock, or at least 100 kg, or at least 500 kg.

Lignocellulosic biomass should generally be pretreated by methods known in the art prior to conducting enzymatic hydrolysis and microbial pre-conditioning. In some embodiments, biomass is pretreated by hydrothermal pretreatment. “Hydrothermal pre-treatment” refers to the use of water, either as hot liquid, vapor steam or pressurized steam comprising high temperature liquid or steam or both, to “cook” biomass, at temperatures of 120° C. or higher, either with or without addition of acids or other chemicals. In some embodiments, ligncellulosic biomass feedstocks are pretreated by autohydrolysis. “Autohydrolysis” refers to a pre-treatment process in which acetic acid liberated by hemicellulose hydrolysis during pre-treatment further catalyzes hemicellulose hydrolysis, and applies to any hydrothermal pre-treatment of lignocellulosic biomass conducted at pH between 3.5 and 9.0.

In some embodiments, hydrothermally pretreated lignocellulosic biomass may be separated into a liquid fraction and a solid fraction. “Solid fraction” and “Liquid fraction” refer to fractionation of pretreated biomass in solid/liquid separation. The separated liquid is collectively referred to as “liquid fraction.” The residual fraction comprising considerable insoluble solid content is referred to as “solid fraction.” Either the solid fraction or the liquid fraction or both combined may be used to practice methods of the invention or to produce compositions of the invention. In some embodiments, the solid fraction may be washed.

### Example 1. Concurrent Microbial Fermentation Improves Organic Capture by Enzymatic Hydrolysis of Unsorted MSW

Laboratory bench scale reactions were conducted with bioliquid sample from the test described in example 5.

The model MSW substrate for laboratory scale reactions was prepared using fresh produce to comprise the organic fraction (defined as the cellulosic, animal and vegetable fractions) of municipal solid waste (prepared as described in Jensen et al., 2010 based on Riber et al. 2009).

The model MSW was stored in aliquots at −20° C. and thawed overnight at 4° C. The reactions were done in 50 ml centrifuge tubes and the total reaction volume was 20 g. Model MSW was added to 5% dry matter (DM) (measured as the dry matter content remaining after 2 days at 60° C.).

The cellulase applied for hydrolysis was Cellic CTec3 (VDNI0003, Novozymes A/S, Bagsvaerd, Denmark) (CTec3). To adjust and maintain the pH at pH5, a citrate buffer (0.05M) was applied to make up the total volume to 20 g.

The reactions were incubated for 24 hours on a Stuart Rotator SB3 (turning at 4 RPM) placed in a heating oven (Binder GmBH, Tuttlingen, Germany). Negative controls were done in parallel to assess background release of dry matter from the substrate during incubation.

Following incubation the tubes were centrifuged at 1350 g for 10 minutes at 4° C. The supernatant was then decanted off, 1 ml was removed for HPLC analysis and the remaining supernatant and pellet were dried for 2 days at 60° C. The weight of dried material was recorded and used to calculate the distribution of dry matter. The conversion of DM in the model MSW was calculated based on these numbers.

The concentrations organic acids and ethanol were measured using an UltiMate 3000 HPLC (Thermo Scientific Dionex) equipped with a refractive index detector (Shodex® RI-101) and a UV detector at 250 nm. The separation was performed on a Rezex RHM monosaccharide column (Phenomenex) at 80° C. with 5 mM H₂SO₄ as eluent at a flow rate of 0.6 ml/min. The results were analyzed using the Chromeleon software program (Dionex).

To evaluate the effect of concurrent fermentation and hydrolysis, 2 ml/20 g of the bioliquid from the test described in example 5 (sampled on December 15^{th} and 16^{th}) was added to the reactions with or without CTec3 (24 mg/g DM).

### Conversion of DM in MSW.

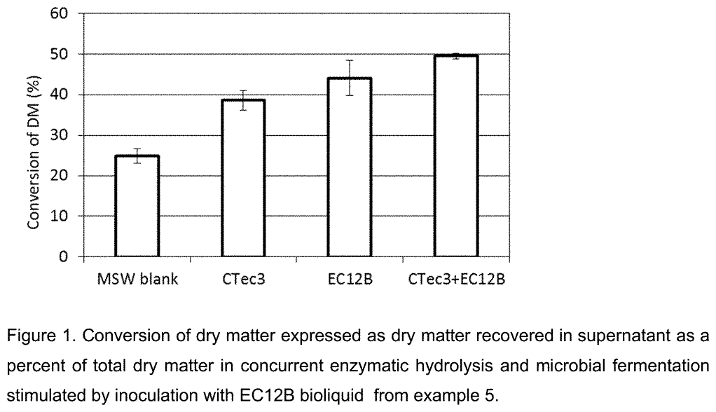

The conversion of solids was measured as the content of solids found in the supernatant as a percent of total dry matter. FIG. 1 shows conversion for MSW blank, isolated enzyme preparation, microbial inoculum alone, and the combination of microbial inoculum and enzyme. The results shows that addition of EC12B from example 5 resulted in significantly higher conversion of dry matter compared to the background release of dry matter in the reaction blank (MSW Blank) (Students t-Test p<0.0001). Concurrent microbial fermentation induced by addition of the EC12B sample and enzymatic hydrolysis using CTec3 resulted in significantly Higher Conversion of Dry Matter Compared to the Reaction Hydrolysed Only with CTec3 and the reactions added EC12B alone (p<0.003).

### HPLC Analysis of Glucose, Lactate, Acetate and EtOH.

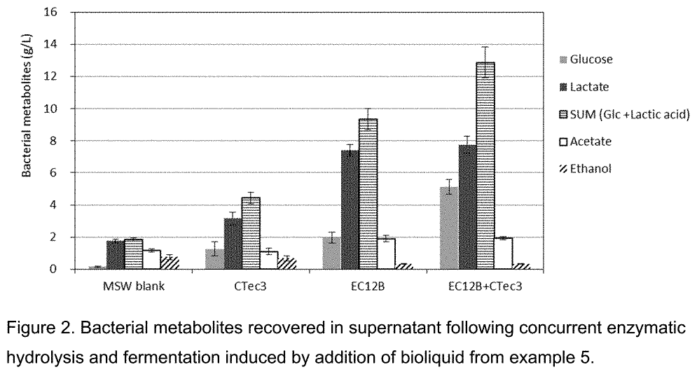

The concentration of glucose and the microbial metabolites (lactate, acetate and ethanol) measured in the supernatant are shown in FIG. 2. As shown, there was a low background concentration of these in the model MSW blank and the lactic acid content presumably comes from bacteria indigenous to the model MSW since the material used to create the substrate was in no way sterile or heated to kill bacteria. The effect of addition of CTec3 resulted in an increase in glucose and lactic acid in the supernatant. The highest concentrations of glucose and bacterial metabolites was found in the reactions where EC12B bioliquid from example 5 was added concurrently with CTec3. Concurrent fermentation and hydrolysis thus improve conversion of dry matter in model MSW and increase the concentration of bacterial metabolites in the liquids.

References: Jacob Wagner Jensen, Claus Felby, Henning Jorgensen, Georg Ørnskov Rønsch, Nanna Dreyer Nørholm. Enzymatic processing of municipal solid waste. Waste Management. December 2010; 30(12):2497-503.

Riber, C., Petersen, C., Christensen, T. H., 2009. Chemical composition of material fractions in Danish household waste. Waste Management 29, 1251-1257.

### Example 2. Concurrent Microbial Fermentation Improves Organic Capture by Enzymatic Hydrolysis of Unsorted MSW

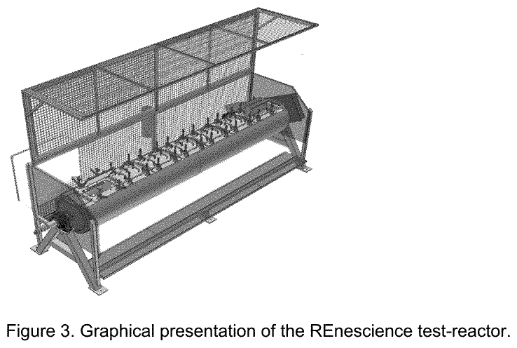

Tests were performed in a specially designed batch reactor shown in FIG. 3, using unsorted MSW with the aim to validate results obtained in lab scale experiments. The experiments tested the effect of adding an inoculum of microorganisms comprising bioliquid obtained from example 3 bacteria in order to achieve concurrent microbial fermentation and enzymatic hydrolysis. Tests were performed using unsorted MSW.

MSW used for small-scale trials were a focal point of the research and development at REnescience. For the results of trials to be of value, waste was required to be representative and reproducible.

Waste was collected from Nomi US Holstebro in March 2012. Waste was unsorted municipal solid waste (MSW) from the respective area. Waste was shredded to 30×30 mm for use in small-scale trials and for collection of representative samples for trials. Theory of sampling was applied to shredded waste by sub-sampling of shredded waste in 22-litre buckets. Buckets were stored in a freezer container at −18° C. until use. “Real waste” was composed of eight buckets of waste from the collection. The content of these buckets was remixed and resampled in order to ensure that variability between repetitions was as low as possible.

All samples were run under similar conditions regarding water, temperature, rotation and mechanical effect. Six chambers were used: three without inoculation and three with inoculation. Designated non-water content during trial was set to 15% non-water content by water addition. Dry matter in the inoculating material was accounted for so the fresh water addition in the inoculated chambers was smaller. 6 kg of MSW was added to each chamber, as was 84 g CTEC3, a commercial cellulase preparation. 2 liter of inouculum was added to inoculated chambers, with a corresponding reduction in added water.

pH was kept at 5.0 in the inoculated chambers and at pH 4.2 in the non-inoculated chambers using respectively addition of 20% NaOH for increasing pH and 72% H₂SO₄for decreasing the pH. The lower pH in the non-inouclated chamber helped ensure that intrinsic bacteria would not flourish. We have previously shown that, using the enzyme preparation used, CTEC3 Tm, in the context of MSW hydrolysis, no difference in activity can be discerned between pH 4.2 and pH 5.0 The reaction was continued at 50 degrees C. for 3 days, with the pilot reactor providing constant rotary agitation.

At the end of the reaction, the chambers were emptied through a sieve and bioliquid comprising liquefied material produced by concurrent enzymatica hydrolysis and microbnial fermentation of MSW.

Dry matter (TS) and volatile solids (VS) were determined Dry Matter (DM) method: Samples were dried at 60° C. for 48 hours. The weight of the sample before and after drying was used to calculate the DM percentage.

$$Sample DM ( \% ) \frac{Sample dry weight}{Wet weight ( g )} \times 100$$

Volatile solids method:

Volatile solids are calculated and presented as the DM percentage subtracted the ash content. The ash content of a sample was found by burning the pre-dried sample at 550° C. in a furnace for a minimum of 4 hours. Then the ash was calculated as:

Sample Ash percentage of dry matter:

$$\frac{Sample ash weight ( g )}{Sample dry weight ( g )} \times 100$$

Volatile Solids percentage:

(1−sample ash percentage)×Sample DM percentage

Results were as shown below. As shown, a higher total solids content was obtained in bioliquid obtained in the inouculated chambers, indicating that concurrent microbial fermentation and enzymatic hydrolysis were superior to enzymatic hydrolysis alone.

Bioliquid

| | TS (kg) | VS (kg) |
|---|---|---|
| Std. low lactate | 1.098 | 0.853 |
| Pode. High lactate | 1.376 | 1.041 |
| Added pode. TS + VS | TS | VS |
| Kg | 0.228 | 0.17 |

Bioliquid

| Produced | TS (kg) | stdev | VS (kg) | stdev |
|---|---|---|---|---|
| std. low lactate | 1.098 | 0.1553 | 0.853 | 0.116 |
| Pode. High lactate | 1.148 | 0.0799 | 0.869 | 0.0799 |
| | more % | | more % | |
| std. low lactate | | | | |
| Pode. High lactate | 4.5579 | | 1.8429 | |

% more

Sum metabolics (lactate acetate and ethanol) produced

| | | | |
|---|---|---|---|
| std avg. | 92.20903 | g/L | |
| pode avg. | 342.6085 | g/L | 271.5564 |

Sum metabolics (lactate acetate and ethanol) “captured”

| | | | |
|---|---|---|---|
| std avg. (low lac) | 189.6075 | g/L | |
| pode avg. (high lac) | 461.6697 | g/L | 143.4871 |

### Example 3. Concurrent Microbial Fermentation Improves Organic Capture by Enzymatic Hydrolysis of Unsorted MSW

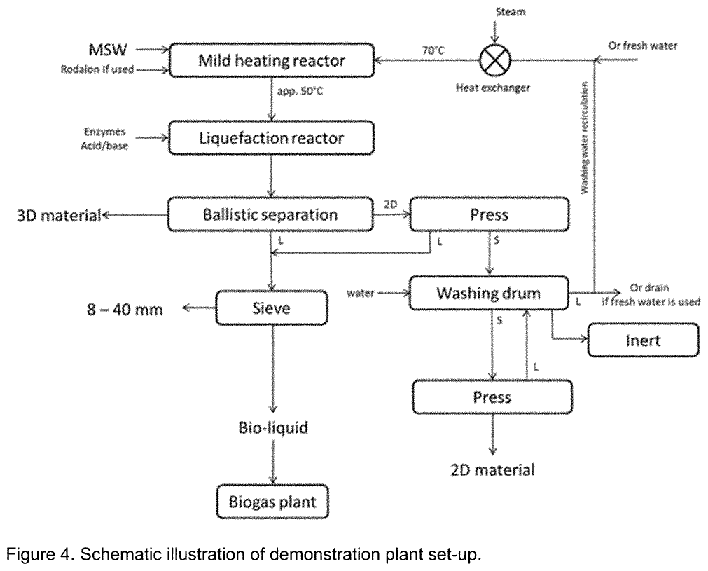

Experiments were conducted at the REnescience demonstration plant placed at Amager ressource center (ARC), Copenhagen, Denmark. A schematic drawing showing principle features of the plant is shown in FIG. 4. The concept of the ARC REnescience Waste Refinery is to sort MSW in to four products. A bio-liquid for biogas production, inerts (glass and sand) for recycling and 2D and 3D fractions of inorganic materials suitable for RDF production or recycling of metals, plastic and tree.

MSW from big cities is collected as is in plastic bags. The MSW is transported to the REnescience Waste Refinery where it is stored in a silo until processing. Depending on the character of the MSW a sorting step can be installed in front of the REnescience system to take out oversize particles (above 600 mm).

REnescience technology as tested in this example comprises three steps. The first step is a mild heating (pretreatment, as shown in FIG. 4) of the MSW by hot water to temperatures in the range of 40-75° C. for a period of 20-60 minutes. This heating and mixing period opens plastic bags and provides adequate pulping of degradable components preparing a more homogenous organic phase before addition of enzymes. Temperature and pH are adjusted in the heating period to the optimum of isolated enzyme preparatons which are used for enzymatic hydrolysis. Hot water can be added as clean tap water or as washing water first used in the washing drums and then recirculated to the mild heating as indicated in FIG. 4.

The second step is enzymatic hydrolysis and fermentation (liquefaction, as shown in FIG. 4). In the second step of the REnescience process enzymes are added and optionally selected microorganisms. The enzymatic liquefaction and fermentation is performed continuously at a residence time of app. 16 hours, at the optimal temperature and pH for enzyme performance. By this hydrolysis and fermentation the biogenic part of the MSW is liquefied in to a bio-liquid high in dry matter in between non-degradable materials. pH is controlled by addition of CaCO₃.

The third step of REnescience technology as practiced in this example is a separation step where the bio-liquid is separated from the non-degradable fractions. The separation is performed in a ballistic separator, washing drums and hydraulic presses. The ballistic separator separates the enzymatic treated MSW into the bio-liquid, a fraction of 2D non-degradable materials and a fraction of 3D non-degradable materials. The 3D fraction (physical 3 dimensional objects as cans and plastic bottles) does not bind large amounts of bio-liquid, so a single washing step is sufficient to clean the 3D fraction. The 2D fraction (textiles and foils as examples) binds a significant amount of bio-liquid. Therefore the 2D fraction is pressed using a screw press, washed and pressed again to optimize the recovery of bio-liquid and to obtain a “clean” and dry 2D fraction. Inert material which is sand and glass is sieved from the bio-liquid. The water used in all the washing drums can be recirculated, heated and then used as hot water in the first step for heating.

The trial documented in this example was split up in three sections as shown in table 1

TABLE 1

| | | Tap water/Washing water |
|---|---|---|
| Time (hours) | Rodalon | to mild heating |
| 27-68 | + | tap water |
| 86-124 | − | tap water |
| 142-187 | − | washing water |

In a 7-day trial, unsorted MSW obtained from Copenhagen, Denmark was loaded continuously by 335 kg/h in to the REnescience demo plant. In the mild heating was added 536 kg/h water (tap water or washing water) heated to app. 75° C. before entering the mild heating reactor. Temperature is hereby adjusted to app. 50° C. in the MSW and pH is adjusted to app. 4.5 by addition of CaCO₃.

In the first section the surface-active anti-bacterial agent Rodalon™ (benzyl alkyl ammonium chloride) was included in the added water at 3 g active ingredient per kg MSW.

In the liquefaction reactor is added app. 14 kg of Cellic Ctec3 (commercially available cellulase preparation from Novozymes) per wet ton of MSW. The temperature was kept in the range from 45-50° C. and the pH was adjusted in the range from 4.2-4.5 by adding CaCO₃. Enzyme reactor retention time is app. 16 hours.

In the separation system of ballistic separator, presses and washing drums the bio-liquid (liquefied degradable material) is separated from non-degradable materials.

Wash waters were selectively either poured out, recording organic content, or recirculated and re-used to wet incoming MSW in the mild heating. Recirculation of wash water has the effect of accomplishing bacterial inoculation using organisms thriving at 50° C. reaction conditions to levels higher than those initially present. In the process scheme used, recirculated wash water were first heated to approximately 70° C., in order to bring incoming MSW to a temperature appropriate for enzymatic hydrolysis, in this case, about 50° C. Particularly in the case of lactic acid bacteria, heating to 70C has previously been shown to provide a selection and “inducement” of thermal tolerance expression.

Samples were obtained at selected time points at the following places:
The bio-liquid leaving the small sieve, which is termed “EC12B”
The bio-liquid in the storage tank
Washing water after the whey sieves
2D fraction
3D fraction
Inert bottom fraction from both washing units

The production of bioliquid was measured with load cells on the storage tank. The input flow of fresh waters was measured with flowmeters, the recycled or drained washing waste was measured with load cells.

Bacterial counts were examined as follows: Selected samples of bioliquid were diluted 10-fold in the SPO (peptone salt solution) and 1 ml of the dilutions are plated at sowing depth on beaf Extract Agar (3.0 g/L of Beef extract (Fluka, Cas.: B4888), 10.0 g/L Tryptone (Sigma, cas.no.: T9410), 5.0 g/L NaCl (Merck, cas.no. 7647-14-5), 15.0 g/L agar (Sigma, cas. no. 9002-18-0)). The plates were incubated at 50 degrees, respectively. aerobic and anaerobic atmosphere. Anaerobic cultivation took place in appropriate containers were kept anaerobic by gassing with Anoxymat and adding iltfjernende letters (AnaeroGen from Oxoid, cat.no AN0025A). The aerobic colonies were counted after 16 hours and again after 24 hours. The anaerobic growing bacteria were quantified after 64-72 hours.

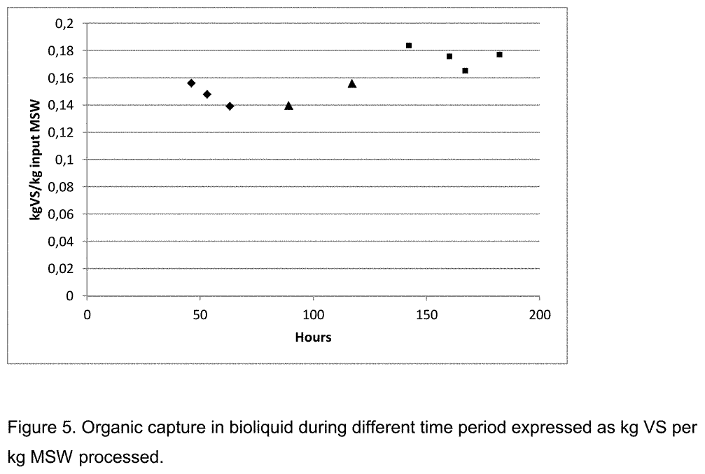

FIG. 5 shows total volatile solids content in bioliquid samples at EC12B as kg per kg MSW processed. Points estimates were obtained at different time points during the experiment by considering each of the three separate experimental periods as a separate time period. Thus, a point estimate during period 1 (Rodalon) is expressed relative to the mass balances and material flows during period 1. A shown in FIG. 5, during period 1, which was initiated after a prolonged stop due to complications in the plant, total solids captured in bioliquid are seen to drop steadily, consistent with a slight anti-bacterial effect of Rodalon™. During period 2, total captured solids returns to slightly higher levels. During period 3, where recirculation provides an effective “inoculation” of incoming MSW, bioliquid kg VS/kg affald rises to considerably higher levels around 12%.

For each of the 10 time points shown in FIG. 5, bioliquid (EC12B) samples were taken and total solids, volatile solids, dissolved voilatile solids, and concentrations of the presumed bacterial metabolites acetate, butyrate, ethanol, formate, and propionate were determined by HPLC. These results including glycerol concentrations are shown in Table 1 below.

TABLE 1
Analysis of bioliquid samples.

| Time | Total solids | VS | Dissolved VS | Lactate | Formic acid | Acetate | Propionate | Ethanol | Glycerol |
|---|---|---|---|---|---|---|---|---|---|
| hours | % | % | % | % | % | % | % | % | % |
| 45 | 10.30 | 8.69 | 7.00 | 3.22 | 0.00 | 0.35 | 0.00 | 0.12 | 0.4165 |
| 53 | 9.77 | 8.22 | 6.62 | 3.00 | 0.00 | 0.42 | 0.00 | 0.17 | 0 |
| 63 | 9.31 | 7.74 | 6.07 | 2.74 | 0.09 | 0.41 | 0.03 | 0.17 | 0.415 |
| 67 | 8.66 | 7.15 | 5.54 | 2.82 | 0.00 | 0.39 | 0.03 | 0.20 | 0.475 |
| 88 | 9.57 | 7.97 | 6.02 | 3.24 | 0.00 | 0.31 | 0.04 | 0.13 | 0.554 |
| 116 | 10.57 | 8.90 | 6.77 | 3.27 | 0.01 | 0.25 | 0.00 | 0.11 | 0.5635 |
| 130 | 9.93 | 8.33 | 6.43 | 3.39 | 0.00 | 0.25 | 0.00 | 0.11 | 0 |
| 141 | 12.07 | 9.08 | 6.76 | 4.16 | 0.00 | 0.28 | 0.00 | 0.14 | 0.6205 |
| 159 | 11.30 | 8.68 | 6.33 | 4.63 | 0.00 | 0.31 | 0.00 | 0.11 | 0 |
| 166 | 11.04 | 8.17 | 5.72 | 4.50 | 0.00 | 0.32 | 0.03 | 0.12 | 0.646 |
| 181 | 11.76 | 8.75 | 6.11 | 5.48 | 0.12 | 0.37 | 0.00 | 0.11 | 1.38 |
| 188 | 11.20 | 8.05 | 6.20 | 5.40 | 0.00 | 0.40 | 0.00 | 0.11 | 0 |

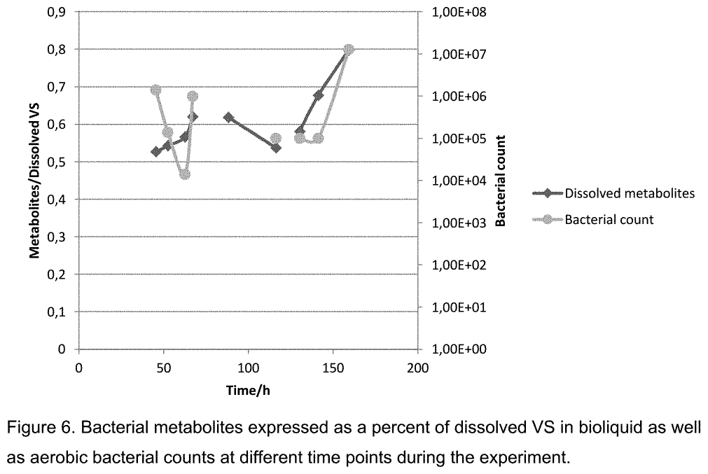

For bioliquid samples taken at each of the ten time points, FIG. 6 shows both live bacterial counts determined under aerobic confitions and also the weight percent “bacterial metabolites” (meaning the sum of acetate, butyrate, ethanol, formate, and proprionate) expressed as a percentage of dissolved volatile solids. As shown, the weight percent bacterial metabolites clearly increases with increased bacterial activity, and is associated with increased capture of solids in the bioliquid.

### Example 4. Identification of Microorganisms Contributing to the Concurrent Fermentation in Example 3

Samples of bioliquid obtained from example 3 were analysed for microbial composition. The microbial species present in the sample were identified by comparing their 16S rRNA gene sequences with 16S rRNA gene sequences of well-characterized species (reference species). The normal cut-off value for species identification is 97% 16S rRNA gene sequence similarity with a reference species. If the similarity is below 97%, it is most likely a different species.

The resulting sequences were queried in a BlastN against the NCBI databasese. The database contains good quality sequences with at least 1200 bp in length and a NCBI taxonomic association. Only BLAST hits ≥95% identity were included.

The sampled bioliquid was directly transferred to analysis without freezing before DNA extraction.

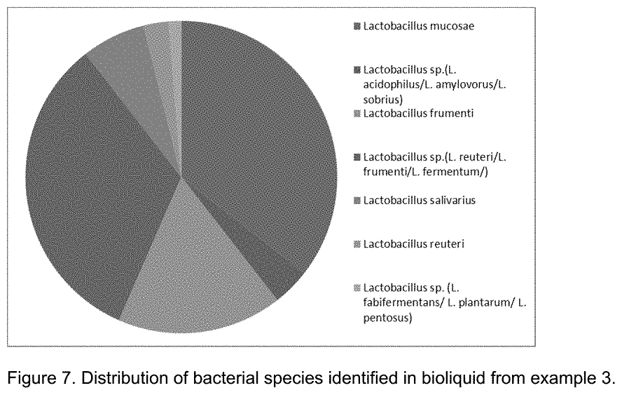

A total of 7 bacterial species were identified (FIG. 7) and 7 species of Archea were identified (FIG. 2). In some cases the bacterial species the subspecies could not be assigned (L. acidophilus, L. amylovorus, L. sobrius, L. reuteri, L. frumenti, L. fermentum, L. fabifermentans, L. plantarum, L. pentosus)

### Example 5. Detailed Analysis of Organic Capture Using Concurrent Microbial Fermentation and Enzymatic Hydrolysis of Unsorted MSW

The REnescience demonstration plant described in example 1 was used to make a detailed study of total organic capture using concurrent bacterial fermentation and enzymatic hydrolysis of unsorted MSW.

Trash from Copenhagen was characterized by Econet to determine its content (method, quantity).

Waste analysis have been analysed to determine the content and variation. A large sample of MSW was delivered to Econet A/S, which performed the waste analyses. The primary sample was reduced to a sub sample around 50-200 kg. This subsample was the sorted by trained personnel into 15 different waste fractions. The weight of each fraction was recorded and a distribution calculated.

TABLE x
Waste composition as (%) of total, analysed by Econet during the 300 hours test

| | Sample: | | | | | | | | | | |
|---|---|---|---|---|---|---|---|---|---|---|---|
| | 1. | 2. | 3. | 4. | 5. | 6. | 7. | 8. | 9. | average | Standard |
| | % | % | % | % | % | % | % | % | % | % | deviation |
| Plastic packaging | 5.1 | 6.7 | 8.0 | 4.9 | 6.2 | 2.5 | 6.2 | 7.5 | 6.4 | 5.9 | 1.64 |
| Plastic foil | 10.8 | 8.6 | 10.7 | 7.9 | 10.1 | 7.8 | 8.8 | 8.5 | 9.5 | 9.2 | 1.13 |
| Other plastic | 0.7 | 0.8 | 0.5 | 0.7 | 1.0 | 0.7 | 1.6 | 0.4 | 0.9 | 0.8 | 0.33 |
| Metal | 2.5 | 3.6 | 2.7 | 2.0 | 2.5 | 2.1 | 3.6 | 2.1 | 3.6 | 2.7 | 0.68 |
| Glass | 0.2 | 0.0 | 0.5 | 0.6 | 0.6 | 0.0 | 0.6 | 0.4 | 0.0 | 0.3 | 0.27 |
| Yard waste | 0.7 | 3.5 | 1.9 | 1.8 | 0.9 | 2.7 | 0.6 | 4.5 | 2.8 | 2.1 | 1.33 |
| WEEE (batteries etc.) | 0.7 | 0.1 | 0.6 | 0.4 | 0.7 | 0.8 | 1.1 | 0.1 | 0.5 | 0.6 | 0.33 |
| Paper | 14.8 | 8.3 | 13.3 | 8.8 | 10.5 | 5.6 | 10.2 | 12.6 | 12.4 | 10.7 | 2.86 |
| Plastic and cardboard packaging | 10.4 | 21.4 | 11.9 | 8.6 | 11.0 | 6.7 | 10.7 | 11.8 | 13.9 | 11.8 | 4.13 |
| Food waste | 19.8 | 15.6 | 25.9 | 27.6 | 26.3 | 24.5 | 24.5 | 23.3 | 18.0 | 22.8 | 4.09 |
| Diapers | 8.0 | 10.3 | 6.9 | 18.8 | 8.1 | 25.1 | 15.2 | 10.1 | 14.0 | 12.9 | 6.00 |
| Dirty paper | 8.5 | 6.7 | 7.3 | 7.4 | 8.5 | 8.6 | 7.9 | 5.7 | 6.3 | 7.4 | 1.03 |
| Fines | 9.7 | 2.5 | 4.2 | 2.1 | 4.5 | 4.7 | 2.7 | 7.0 | 4.9 | 4.7 | 2.40 |
| Other combustibles | 2.0 | 0.9 | 0.8 | 1.2 | 1.8 | 0.7 | 0.7 | 2.2 | 0.8 | 1.2 | 0.61 |
| Other non-combustibles | 6.2 | 11.1 | 5.0 | 7.3 | 7.2 | 7.6 | 5.6 | 3.7 | 6.2 | 6.7 | 2.07 |
| sum | 100 | 100 | 100 | 100 | 100 | 100 | 100 | 100 | 100 | 100.0 | |

The composition of waste varies from time to time, presented in table 2 is waste analysis result from different samples collected over 300 hours. the larges variation is seen en the fractions diapers plastic and cardboard packing and food waste which is all fractions that affect the content of organic material that can be captured.

Over the entire course of the “300 Hours Test,” the average “captured” biodegradable material expressed as kg VS per kg MSW processed was 0.156 kg VS/kg MSW input.

Representative samples of bioliquid were taken at various time points during the course of the experiment, when the plant was in a period of stable operation. Samples were analysed by HPLC and to determine volatile solids, total solids, and dissolved solids as described in example 3. Results are shown in Table 2 below.

TABLE 2
Analysis of bioliquid samples.

| Time | Total solids | VS | Dissolved VS | Formic acid | Lactate | Acetate | Propionate | Ethanol | Glycerol |
|---|---|---|---|---|---|---|---|---|---|
| hours | % | % | % | % | % | % | % | % | % |
| 212 | 10.45 | 8.36 | 5.95 | 0.00 | 5.36 | 0.46 | 0.03 | 0.46 | 0.82 |
| 239 | 10.91 | 8.64 | 5.85 | 0.00 | 6.08 | 0.33 | 0.00 | 0.33 | 0.77 |
| 264.5 | 11.35 | 8.82 | 6.25 | 0.00 | 4.97 | 0.49 | 0.00 | 0.49 | 1.06 |
| 294 | 10.66 | 8.48 | 5.60 | 0.08 | 3.37 | 0.39 | 0.00 | 0.39 | 0.55 |

### Example 6. Identification of Microorganisms Contributing to Concurrent Fermentation in Example 5

A sample of the bioliquid “EC12B” was withdrawn during the test described in example 5 on December the 15^{th} and 16^{th} 2012 and stored at −20° C. for the purpose of performing 16S rDNA analysis to identify the microorganisms in the sample. The 16S rDNA analysis is widely used to identification and phylogenic analysis of prokaryotes based on the 16S component of the small ribosomal subunit. The frozen samples were shipped on dry ice to GATC Biotech AB, Solna, SE where the 16S rDNA analysis was performed (GATC_Biotech). The analysis comprised: extraction of genomic DNA, amplicon library preparation using the universal primers primer pair spanning the hypervariable regions V1 to V3 27F: AGAGTTTGATCCTGGCTCAG (SEQ ID NO: 1)/534R: ATTACCGCGGCTGCTGG (SEQ ID NO: 2); 507 bp length), PCR tagging with GS FLX adaptors, sequencing on a Genome Sequencer FLX instrument to obtain 104.000-160.000 number of reads pr. sample. The resulting sequences were then queried in a BlastN against the rDNA database from Ribosomal Database Project (Cole et al., 2009). The database contains good quality sequences with at least 1200 bp in length and a NCBI taxonomic association. The current release (RDP Release 10, Updated on Sep. 19, 2012) contains 9,162 bacteria and 375 archaeal sequences. The BLAST results were filtered to remove short and low quality hits (sequence identity ≥90%, alignment coverage ≥90%).

A total of 226 different bacteria were identified.

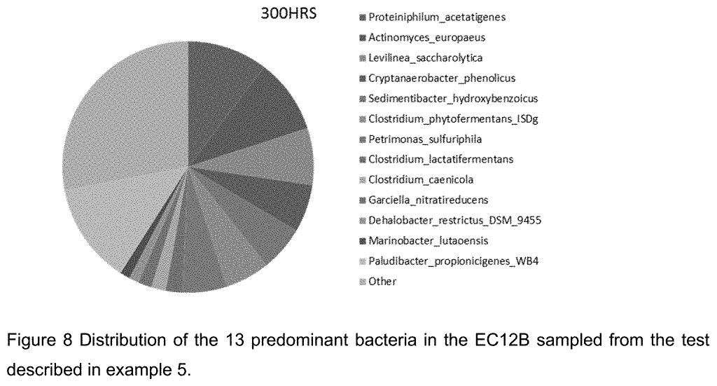

The predominant bacteria in the EC12B sample was Paludibacter propionicigenes WB4, a propionate producing bacteria (Ueki et al. 2006), which comprised 13% of the total bacteria identified. The distribution of the 13 predominant bacteria identified (Paludibacter propionicigenes WB4, Proteiniphilum acetatigenes, Actinomyces europaeus, Levilinea saccharolytica, Cryptanaerobacter phenolicus, Sedimentibacter hydroxybenzoicus, Clostridium phytofermentans ISDg, Petrimonas sulfuriphila, Clostridium lactatifermentans, Clostridium caenicola, Garciella nitratireducens, Dehalobacter restrictus DSM 9455, Marinobacter lutaoensis) is shown in FIG. 8.

Comparing the bacteria identified at genus level showed that Clostridium, Paludibacter, Proteimphilum, Actinomyces and Levilinea (all anaerobes) represented approximately half of the genera identified. The genus Lactobacillus comprised 2% of the bacteria identified. The predominant bacterial specie P. propionicigenes WB4 belong to the second most predominating genera (Paludibacter) in the EC12B sample.

The predominant pathogenic bacteria in the EC12B sample was Streptococcus spp., which comprised 0.028% of the total bacteria identified. There was not found any spore forming pathogenic bacteria in the bio-liquid.

Streptococcus spp. was the only pathogenic bacteria present in the bio-liquid in example 5. Streptococcus spp. is the bacteria with the highest temperature tolerance (of the non-spore forming) and D-value, which indicates that the amount of time needed at a given temperature to reduce the amount of living Streptococcus spp. cells tenfold, is higher than any of the other pathogenic bacteria reported by Déportes et al. (1998) in MSW. These results show that the conditions applied in example 5 are able to sanitize MSW during sorting in the REnescience process to a level where only Streptococcus spp. was present.

The competition between organism for nutrients, and the increased in temperature during the process will decrease the number of pathogenic organisms significantly and as shown above eliminate presence of pathogens in MSW sorted in the REnescience process. Other factors like pH, a_{w}, oxygen tolerance, CO₂, NaCl, and NaNO₂ also influence growth of pathogenic bacteria in bio-liquid. The interaction between the above mentioned factors, might lower the time and temperature needed to reduce the amount of living cells during the process.

### Example 7. Detailed Analysis of Organic Capture Using Concurrent Microbial Fermentation and Enzymatic Hydrolysis of Unsorted MSW Obtained from a Distant Geographic Location

The REnescience demonstration plant described in example 3 was used to process MSW imported from the Netherlands. The MSW wsas found to have the following composition:

TABLE Y
waste composition (5) of total, analysed by
Econet during the van Gansewinkel test.

| | % |
|---|---|
| Plastic packaging | 5 |
| Plastic foil | 7 |
| Other plastic | 2 |
| Metal | 4 |
| Glass | 4 |
| Yard waste | 4 |
| WEEE (batteries etc) | 1 |
| Paper | 12 |
| Cardboard | 12 |
| Diapers | 4 |
| Dirty paper | 2 |
| Other combustibles | 15 |
| Other non-combustibles | 5 |
| Food waste | 13 |
| Fines | 9 |
| Total | 100 |

The material was subject to concurrent enzymatic hydrolysis and microbial fermentation as described in example 3 and 5 and tested for a plant run of 3 days. Samples of bioloiquid obtained at various time points were obtained and characterized. Results are shown in Table 3.

TABLE 3
Analysis of bioliquid.

| Time | Total solids | VS | Dissolved VS | Lactate | Formic acid | Acetate | Propionate | Ethanol | Glycerol |
|---|---|---|---|---|---|---|---|---|---|
| hours | % | % | % | % | % | % | % | % | % |
| 76 | 7.96 | 6.08 | 3.07 | 4.132 | 0.08 | 0.189 | 0 | 0.298 | 0.4205 |
| 95 | 9.19 | 6.99 | 6.66 | 6.943 | 0 | 0.352 | 0.034 | 0.069 | 0.6465 |

The dissolved VS has been corrected with 9% according to loss of lactate during drying.

### Example 8. Biomethane Production Using Bioliquid Obtained from Concurrent Microbial Fermentation and Enzymatic Hydrolysis of Unsorted MSW

Bioliquid obtained in the experiment described in example 5 was frozen in 20 liter buckets and stored at −18° C. for later use. This material was tested for biomethane production using two identical well prepared fixed filter anaerboic digestion systems comprising an anaerobic digestion consortium within a biofilm immobilized on the filter support.

Initial samples were collected for both the feed and the liquid inside the reactor. VFA, tCOD, sCOD, and ammonia concentrations are determined using HACH LANGE cuvette tests with a DR 2800 Spectrophotometer and detailed VFAs were determined daily by HPLC. TSVS measurements are also determined by the Gravimetric Method.

Gas samples for GC analysis are taken daily. Verification of the feed rate is performed by measuring headspace volume in the feed tank and also the amount of effluent coming out of the reactor. Sampling during the process was performed by collecting with a syringe of liquid or effluent.”

Stable biogas production was observed using both digester systems for a period of 10 weeks, corresponding to between 0.27 and 0.32 L/g CO₂, or between R and Z L/g VS.

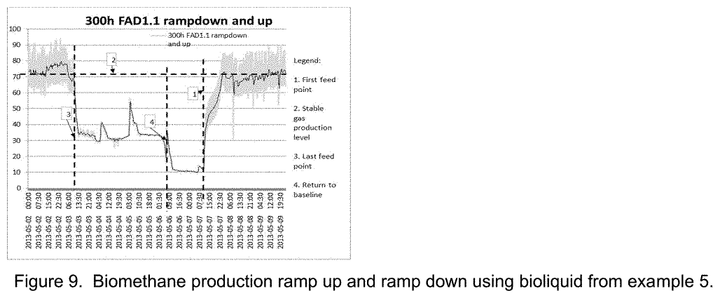

Feed of bioliquid was then discontinued on one of the two system and the return to baseline monitored, as shown in FIG. 9. Stable gas production level is shown by the horizontal line indicated as 2. The time point at which feed was discontinued is shown at the vertical lines indicated as 3. As shown, after months of steady operation, there remained a residual resilient material which was converted during the period indicated between the vertical lines indicated as 3 and 4. The return to baseline or “ramp down” is shown in the period following the vertical line indicated as 4. Following a baseline period, feed was again initiated at the point indicated by the vertical line indicated as 1. The rise to steady state gas production or “ramp up” is shown in the period following the vertical line indicated as 1.

Parameters of gas production from the bioliquid, including “ramp up” and “ramp down” measured as described are shown below.

| | | Sample name |
|---|---|---|
| | | 300 hour |
| Parameter | Unit | Amager waste |
| Feed rate | L/day | 1.85 |
| Total feed | Liter | 3.7 |
| Ramp-up time * | Hours | 15 |
| Ramp-down time ** | Hours | 4 |
| Burn-down time *** | Days | 4 |
| Gas production in stable phase **** | L/day | 122 |
| Total gas produced | L | 244 |
| CH₄ % | % | 60 |
| Total yield | Lgas/Lfeed | 66 |
| Gas from the easy convertible organics | % | 53 |
| Feed COD | g/L | 124 |
| Total COD feed-in | g | 459 |
| COD yield | Lgas/gCOD | 0.53 |
| Specific COD yield | L CH₄/gCOD | 0.32 |
| COD accounted for by mass balance | % of feed COD | 96 |
| COD to gas | g | 418 |
| COD to gas | % | 91 |

* Ramp-up time is the time from first feed till gas production seize to increase and stabilises. The ramp-up time indicates the level of easy convertible organics in the feed.
** Ramp-down time is the time from last feed till gas production seizes to fall steeply. The ramp-down time shows the gas production from easily convertible organics.
*** Burn-down is the time after the Ramp-down time until the gas production seizes totally at base level. The burn-down time shows the gas production from slowly convertible organics.
**** Corrected for background gas production of 2 L/day.

### Example 9. Comparative Biomethane Production Using Bioliquid Obtained from Enzymatic Hydrolysis of Unsorted MSW with and without Concurrent Microbial Fermentation

“High lactate” and “low lactate” bioliquid obtained in example 2 were compared for biomethane production using the fixed filter anaerobic digestion system described in example 8. Measurements were obtained and “ramp up” and “ramp down” times were determined as described in example 8.

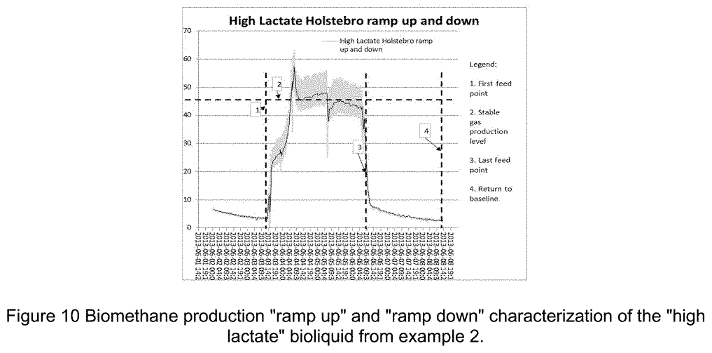

FIG. 10 shows “ramp up” and “ramp down” characterization of the “high lactate” bioliquid. Stable gas production level is shown by the horizontal line indicated as 2. The time point at which feed was initiated is shown at the vertical lines indicated as 1. The rise to steady state gas production or “ramp up” is shown in the period following the vertical line indicated as 1. The time point at which feed was discontinued is shown at the vertical line indicated as 3. The return to baseline or “ramp down” is shown in the period following the vertical line indicated as 3 to the period at the vertical line indicated by 4.

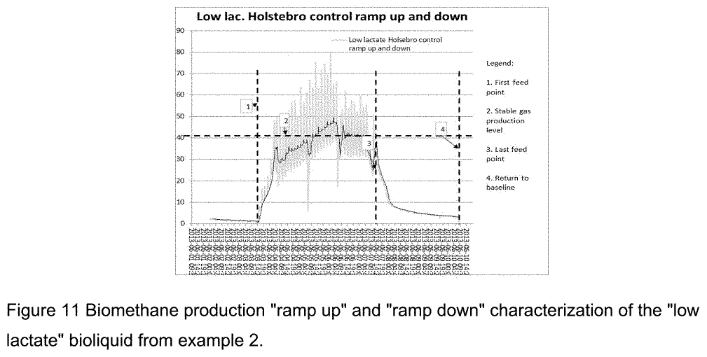

FIG. 11 shows the same characterization of the “low lactate” bioliquid, with the relevant points indicated as described for FIG. 11.

Comparative parameters of gas production from the “high lactate” and “low lactate” bioliquid, including “ramp up” and “ramp down” measured as described are shown below.

The difference in “ramp up”/“ramp down” times show differences in ease of biodegradability. The fastest bioconvertible biomasses will ultimately have the highest total organic conversion rate in a biogas production application. Moreover, the “faster” biomethane substrates are more ideally suited for conversion by very fast anaerobic digestion systems, such as fixed filter digesters.

As shown, the “high lactate” bioliquid exhibits a much faster “ramp up” and “ramp down” time in biomethane production.

| | | Sample name | |
|---|---|---|---|
| | | High | Low |
| | | lactate | lactate |
| | | Holstebro | control |
| Parameter | Unit | waste | Holstebro |
| Feed rate | L/day | 1.0 | 1.0 |
| Total feed | Liter | 2.83 | 3.95 |
| Ramp-up time * | Hours | 16 | 48 |
| Ramp-down time ** | Hours | 6 | 14 |
| Burn-down time *** | Days | 2 | 2 |
| Gas production in stable | L/day | 59 | 40 |
| phase **** | | | |
| Total gas produced | L | 115 | 140 |
| CH₄ % | % | 60 | 60 |
| Total yield | Lgas/Lfeed | 41 | 35 |
| Gas from the easy convertible | % | 86 | 82 |
| organics | | | |
| Feed COD | g/L | 106 | 90 |
| Total COD feed-in | g | 300 | 356 |
| COD yield | Lgas/gCOD | 0.38 | 0.39 |
| Specific COD yield | L CH₄/gCOD | 0.23 | 0.24 |
| COD accounted for by mass | % of feed COD | 91 | 95 |
| balance | | | |
| COD to gas | g | 197 | 240 |
| COD to gas | % | 66 | 68 |

* Ramp-up time is the time from first feed till gas production seize to increase and stabilises. The ramp-up time indicates the level of easy convertible organics in the feed.
** Ramp-down time is the time from last feed till gas production seizes to fall steeply. The ramp-down time shows the gas production from easily convertible organics.
*** Burn-down is the time after the Ramp-down time until the gas production seizes totally at base level. The burn-down time shows the gas production from slowly convertible organics.
**** Corrected for background gas production of 2 L/day.

### Example 11. Biomethane Production Using Bioliquid Obtained from Concurrent Microbial Fermentation and Enzymatic Hydrolysis of Hydrothermally Pretreated Wheat Straw

Wheat straw was pretreated (parameters), separated into a fiber fraction and a liquid fraction, and then the fiber fraction was separately washed. 5 kg of washed fiber were then incubated in a horizontal rotary drum reactor with dose of Cellic CTEC3 with an inoculum of fermenting microorganisms consisting of biovæske obtained from example 3. The wheat straw was subject to simultaneous hydrolysis and microbial fermentation for 3 days at 50 degrees.

This bioliquid was then tested for biomethane production using the fixed filter anaerobic digestion system described in example 8. Measurements were obtained for “ramp up” time as described in example 8.

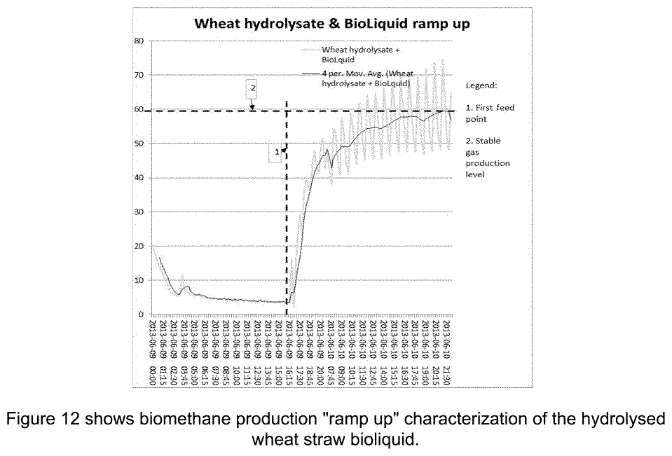

FIG. 12 shows “ramp up” characterization of the hydrolysed wheat straw bioliquid. Stable gas production level is shown by the horizontal line indicated as 2. The time point at which feed was initiated is shown at the vertical lines indicated as 1. The rise to steady state gas production or “ramp up” is shown in the period following the vertical line indicated as 1.

Parameters of gas production from wheat straw hydrolysate bioliquid are shown below.

As shown, pretreated lignocellulosic biomass can also readily be used to practice methods of biogas production and to produce novel biomethane substrates of the invention.

| | | Sample name |
|---|---|---|
| | | Wheat |
| | | hydrolysate + |
| Parameter | Unit | Bioliquid |
| Feed rate | L/day | 1 |
| Total feed | Liter | 1.2 |
| Ramp-up time * | Hours | 29 |
| Ramp-down time ** | Hours | N/A |
| Burn-down time *** | Days | N/A |
| Gas production in stable phase **** | L/day | 56 |
| Total gas produced | L | N/A |
| CH₄ % | % | 60 |
| Total yield | Lgas/Lfeed | N/A |
| Gas from the easy convertible organics | % | N/A |
| Feed COD | g/L | 144 |
| Total COD feed-in | g | 173 |
| COD yield | Lgas/gCOD | N/A |
| Specific COD yield | L CH₄/gCOD | N/A |
| COD accounted for by mass balance | % of feed COD | N/A |
| COD to gas | g | N/A |
| COD to gas | % | N/A |

* Ramp-up time is the time from first feed till gas production seize to increase and stabilises. The ramp-up time indicates the level of easy convertible organics in the feed.
** Ramp-down time is the time from last feed till gas production seizes to fall steeply. The ramp-down time shows the gas production from easily convertible organics.
*** Burn-down is the time after the Ramp-down time until the gas production seizes totally at base level. The burn-down time shows the gas production from slowly convertible organics.
**** Corrected for background gas production of 2 L/day.

### Example 12. Concurrent Microbial Fermentation and Enzymatic Hydrolysis of MSW Using Selected Organisms

The concurrent microbial and enzymatic hydrolysis reactions using specific, monoculture bacteria were carried out in laboratory scale using model MSW (described in example 1) and the procedure described in following the procedure in example 1. The reaction conditions and enzyme dosage are specified in Table 1.

Live bacterial strains of Lactobaccillus amylophiles (DSMZ No. 20533) and propionibacterium acidipropionici (DSMZ No. 20272) (DSMZ, Braunsweig, Germany) (stored at 4° C. for 16 hours until use) were used as inoculum to determine the effect of these on the conversion of dry matter in model MSW with or without addition of CTec3. The major metabolites produced by these are lactic acid and propionic acid, respectively. The concentration of these metabolites were detected using the HPLC procedure (described in example 1).

Since propionibacterium acidipropionici is an anaerobe, the buffer applied in the reactions were this strain was applied, was purged using gaseous nitrogen and the live culture was inoculated to the reaction tubes inside a mobile anaerobic chamber (Atmos Bag, Sigma Chemical CO, St. Louis, Mo., US) also purged with gaseous nitrogen. The reaction tubes with P. propionici were closed before transferred to the incubator. The reactions were inoculated with 1 ml of either P. propionici or L. amylophilus.

The results displayed in table 1 clearly show that the expected metabolites were produced; propionic acid was detected in the reactions inoculated with p. acidipropionic while propionic acid was not detected in the control containing model MSW with or without CTec3. The concentration of lactic acid in the control reaction added only model MSW was almost the same as in the reactions added only L. amylophilus. The production of lactic acid in this control reaction is attributed to bacteria indigenous to the model MSW. Some background bacteria were expected since the individual components of the model waste were fresh produce, frozen, but not further sterilised in any way before preparation of the model MSW. When L. amylophilus was added concurrently with CTec3, the concentration of lactic acid was almost doubled (Table1).

The positive effect on release of DM to the supernatant following hydrolysis was demonstrated as a higher DM conversion in the reactions added either L. amylophilus or P. propionici in conjunction with CTec3 (30-33% increase compared to the reactions added only CTec3).

TABLE 4
Bacterial cultures tested in lab scale alone or concurrently with enzymatic hydrolysis.
The temperature, pH and CTec3 dosage 96 mg/g is shown. Control reactions with
MSW in buffer with or without CTec3 were done in parallel to evaluate the background
of bacterial metabolites in reaction. (Average and standard deviation of 4 reactions
are shown except for the MSW control which were done as singles).

| | | | | Conversion | Propionic | Lactic acid |
|---|---|---|---|---|---|---|
| Temperature | pH | Organism | CTec3 | of DM | acid (g/L) | (g/L) |
| 30° C. | 7 | Propionibacterium | 96 mg/g DM | 17.0 ± 1.0 | 6.2 ± 1.8 | |
| | | acidipropionici | | 40.8 ± 2.2 | 3.7 ± 0.09 | |
| | | MSW control | 96 mg/g DM | 21 | Nd. | |
| | | | | 30.6 | Nd. | |
| | 6.2 | Lactobacillus | 96 mg/g DM | 19.7 ± 2.2 | | 8.4 ± 0.8 |
| | | amylophilus | | 41.7 ± 6.5 | | 21.2 ± 0.7 |
| | | MSW control | 96 mg/g DM | 21 | | 10.3 |
| | | | | 32 | | 16.9 |

Nd. Not detected, below detection limit.

### Example 13. Identification of Microorganisms Contributing to Concurrent Fermentation in Example 7

Samples of the bioliquid “EC12B” and of the recirculated water “EA02” were taken during the test described in example 7 (sampling was done on March 21^{st} and 22^{nd}). The liquid samples were frozen in 10% glycerol and stored at −20° C. for the purpose of performing 16S rDNA analysis to identify the microorganisms in the which is widely used to identification and phylogenic analysis of prokaryotes based on the 16S component of the small ribosomal subunit. The frozen samples were shipped on dry ice to GATC Biotech AB, Solna, SE where the 16S rDNA analysis was performed (GATC_Biotech). The analysis comprised: extraction of genomic DNA, amplicon library preparation using the universal primers primer pair spanning the hypervariable regions V1 to V3 27F: AGAGTTTGATCCTGGCTCAG (SEQ ID NO: 1)/534R: ATTACCGCGGCTGCTGG (SEQ ID NO: 2); 507 bp length), PCR tagging with GS FLX adaptors, sequencing on a Genome Sequencer FLX instrument to obtain 104.000-160.000 number of reads pr. sample. The resulting sequences were then queried in a BlastN against the rDNA database from Ribosomal Database Project (Cole et al., 2009). The database contains good quality sequences with at least 1200 bp in length and a NCBI taxonomic association. The current release (RDP Release 10, Updated on Sep. 19, 2012) contains 9,162 bacteria and 375 archaeal sequences The BLAST results were filtered to remove short and low quality hits (sequence identity ≥90%, alignment coverage ≥90%).

In the samples EC12B-21/3, EC12B-22/3 and EA02B 21/3, EA02-22/3 a total of 452, 310, 785, 594 different bacteria were identified.

The analysis clearly showed, at a species level, that Lactobacillus amylolyticus was by far the most dominating bacterium accounting for 26% to 48% of the entire microbiota detected. The microbiota in the EC12B samples was similar; the distribution of the 13 predominant bacteria (Lactobacillus amylolyticus DSM 11664, Lactobacillus delbrueckii subsp. delbrueckii, Lactobacillus amylovorus, Lactobacillus delbrueckii subsp indicus, Lactobacillus similis JCM 2765, Lactobacillus delbrueckii subsp. Lactis DSM 20072, Bacillus coagulans, Lactobacillus hamsteri, Lactobacillus parabuchneri, Lactobacillus plantarum, Lactobacillus brevis, Lactobacillus pontis, Lactobacillus buchneri) was practically the same comparing the two different sampling dates.

The EA02 samples were similar to the EC12B although L. amylolyticus was less dominant. The distribution of the 13 predominant bacteria (Lactobacillus amylolyticus DSM 11664, Lactobacillus delbrueckii subsp delbrueckii, Lactobacillus amylovorus, Lactobacillus delbrueckii subsp. Lactis DSM 20072, Lactobacillus similis JCM 2765, Lactobacillus delbrueckii subsp. indicus, Lactobacillus paraplantarum, Weissella ghanensis, Lactobacillus oligofermentans LMG 22743, Weissella beninensis, Leuconostoc gasicomitatum LMG 18811, Weissella soli, Lactobacillus paraplantarum) was also similar with the exception of the presence of with the exception of the occurrence of Pseudomonas extremaustralis 14-3 in the 13 predominant bacterial species. This Pseudomonas found in EA02 (21/3) has previously been isolated from a temporary pond in Antarctica and should be able to produce polyhydroxyalkanoate (PHA) from both octanoate and glucose (Lopez et al. 2009; Tribelli et al., 2012).

Comparing the results at a genus level showed that lactobacillus comprised 56-94% of the bacteria identified in the samples Again the distribution across genera is extremely similar between the two sampling dates of EC12B and EA02. Interestingly, in the EA02 samples the genera Weisella, Leuconostoc and Pseudomonas are present to large extent (1.7-22%) while these are only found as minor constituents of the EC12B sample (>0.1%). Weisella and Leuconostoc both belong to the order lactobacillales, the same as the lactobacillus.

The predominant pathogenic bacteria in the EC12B and EA02 sampled during the test described in example 7 comprised 0.281-0.539% and 0.522-0.592%, respectively of the total bacteria identified. The predominant pathogenic bacteria in the EC12B samples were Aeromonas spp., Bacillus cereus, Brucella sp., Citrobacter spp., Clostridium perfrigens, Klebsiells sp., Proteus sp., Providencia sp., Salmonella spp., Serratia sp., Shigellae spp. and Staphylococcus aureus (see FIG. 3). No spore forming pathogenic bacteria were identified in the EC12B and EA02 described in example 7. The total amount of pathogen bacteria identified in both EC12B and EA02 was reduced during time, almost dismissing the amount of total bacteria in EC12B in one day.

In Déportes et al. (1998) an overview of the pathogens know to be present in MSW was made. The pathogens present in the MSW described in examples 3, 5 and 7 are shown in Table 1 (Déportes et al. (1998) and 16S rDNA analysis). In addition to the pathogens described by Déportes et al. (1998), Proteua sp. and Providencia sp. were both found in EC12B and EA02 sampled during the test described in example 7. Whereas the Streptococcus spp. the only pathogenic bacteria present in the bio-liquid in example 5, was not present here. This indicate that another bacterial community is present in EC12B and EA02 in example 7, which might be due to competition between organism for nutrients, and a slight decrease in temperature during the process which will favor the growth of another bacteria community.

TABLE 5
Overview of pathogens present in examples 3, 5 and 7

| Organism | | Max | Temperature | Time req. | D-value | pH range | |
|---|---|---|---|---|---|---|---|
| Bacteria | Optimal | (growth) | Bacteriosidal | [min] | [min] | Min | Max |
| Aeromonas sp. | 37 | 55 | 55 | | 0.25 | | |
| Bacillus cereus | 37 | 50 | 95 | 10 | | 4.8 | 9.3 |
| Brucella sp. | | | | | | | |
| Citrobacter sp. | | | 52.5 | 7 | | 4-5 | |
| Clostridium perfringens | 37 | 50 | 61 | 23 | | 5 | 8.5 |
| Klebsiella sp. | | | 55 | | 0.5 | <3 | |
| Salmonella sp. | 37 | 45 | 55 | 2.5 | | 3.7 | 9.5 |
| Serratia sp. | | | 55 | | 1.5 | | |
| Shigellae spp. | 37 | 48 | 60 | | 1 | 5 | 8 |
| Staphylococcus aureus | | 47.8 | | | | 4 | 9 |
| Streptococcus spp | | | 65 | 20 | | | |

| Organism | aw | Bio | Sources | Ref on growing |
|---|---|---|---|---|
| Bacteria | Min | safetylevel | Found in MSW | condition |
| Aeromonas sp. | 0.94 | 1-2 | (Déportes, et al. 1998) | Rouf and Rigney 1971, |
| | | | | Spinks et al 2006, |
| | | | | Santos et al 1994 |
| Bacillus cereus | 0.951 | 2 | (Déportes, et al. 1998) | Lanciotti et al 2001 |
| Brucella sp. | | 3 | (Déportes, et al. 1998) | |
| Citrobacter sp. | 0.94 | 1-2 | (Déportes, et al. 1998) | Verrips and Kwaps |
| | | | | 1977, Smith and |
| | | | | Bhagwat 2013, Colavita |
| | | | | et al 2003 |
| Clostridium perfringens | 0.95 | 2 | (Déportes, et al. 1998) | Jay, J. M. 1991 |
| Klebsiella sp. | | 1-2 | (Déportes, et al. 1998) | |
| Salmonella sp. | 0.94 | 2-3 | (Déportes, et al. 1998) | Jay, J. M. 1991, |
| | | | | Spinks et al 2006 |
| Serratia sp. | | 2 | (Déportes, et al. 1998) | Spinks et al 2006 |
| Shigellae spp. | — | 2-3 | (Déportes, et al. 1998) | Spinks et al 2006 |
| Staphylococcus aureus | 0.86 | 2 | (Déportes, et al. 1998) | Jay, J. M. 1991 |
| Streptococcus spp | | 2 | (Déportes, et al. 1998) | Francis, A. E. 1959 |

### Strain Identification and DSMZ Deposits

Samples of EA02 from March 21^{st} and 22^{nd} retrieved from the test described in example 7, were sent for plating at the Novo Nordic Centre for Biosustainability (NN Center)(Hoersholm, Denmark) with the purpose of identifying and obtaining monocultures of isolated bacteria. Upon arrival at the NN center, the samples were incubated overnight at 50° C., then plated on different plates (GM17, tryptic soy broth, and beef extract (GM17 agar: 48.25 g/L m17 agar, after 20 min. autoclaving added Glucose to final concentration at 0.5%, Tryptic soy agar: 30 g/L Tryptic soy broth, 15 g/L agar, Beef broth (Statens Serum Institute, Copenhagen, Denmark) added 15 g/l agarose) and grown aerobically at 50° C.

After one day, the plates were visually inspected and selected colonies were re-streaked on the corresponding plates and send to DSMZ for identification.

The following strains isolated from the recirculated water from EA02 have been put in patent deposit at DMSZ, DSMZ, Braunsweig, Germany:

### Identified Samples

Sample ID: 13-349 (Bacillus safensis) originating from (EA02-21/3), DSM 27312

Sample ID: 13-352 (Brevibacillus brevis) originating from (EA02-22/3), DSM 27314

Sample ID: 13-353 (Bacillus subtilis sp. subtilis) originating from (EA02-22/3), DSM 27315

Sample ID: 13-355 (Bacillus licheniformis) originating from (EA02-21/3), DSM 27316

Sample ID: 13-357 (Actinomyces bovis) originating from (EA02-22/3), DSM 27317

### Not Identified Samples

Sample ID: 13-351 originating from (EA02-22/3), DSM 27313

Sample ID: 13-362A originating from (EA02-22/3), DSM 27318

Sample ID: 13-365 originating from (EA02-22/3), DSM 27319

Sample ID: 13-367 originating from (EA02-22/3), DSM 27320

### REFERENCES

Cole, J. R., Wang, Q., Cardenas, E., Fish, J., Chai, B., Farris, R. J., & Tiedje, J. M. (2009). The Ribosomal Database Project: improved alignments and new tools for rRNA analysis. Nucleic acids research, 37 (suppl 1), (D141-D145).
GATC_Biotech supporting material. Defining the Microbial Composition of Environmental Samples Using Next Generation Sequencing. Version 1.
Tribelli, P. M., Iustman, L. J. R., Catone, M. V., Di Martino, C., Reyale, S., Mendez, B. S., Lopez, N. I. (2012). Genome Sequence of the Polyhydroxybutyrate Producer Pseudomonas extremaustralis, a Highly Stress-Resistant Antarctic Bacterium. J. Bacteriol. 194(9):2381.
Nancy I. Lopéz, N. I., Pettinari, J. M., Stackebrandt, E., Paula M. Tribelli, P. M., Pötter, M., Steinbüchel, A., Mendez, B. S. (2009). Pseudomonas extremaustralis sp. nov., a Poly(3-hydroxybutyrate) Producer Isolated from an Antarctic Environment. Cur. Microbiol. 59(5):514-519.

The embodiments and examples are representative only and not intended to limit the scope of the claims.

1. A method for increasing the organic yield of a bioliquid, said method comprising, Inoculating waste comprising collected domestic household waste, wastes from restaurants and food processing facilities, and/or wastes from office buildings with one or more species of lactic acid bacteria, acetate-producing bacteria, propionate-producing bacteria, or butyrate-producing bacteria and fermenting said inoculated waste at a temperature between 35-75° C. wherein said one or more species of bacteria used for inoculating the waste do not produce ethanol as the primary product of fermentation, and concurrently enzymatically treating the waste with cellulase enzymes to release biodegradable parts of the waste into a liquid fraction; and
removing the non-biodegradable solids to produce a bioliquid wherein lactate is present in a higher concentration by weight in the bioliquid when compared to any other single dissolved volatile solid selected from the group consisting of: acetate, butyrate, ethanol, formate, and/or propionate, and
wherein said organic yield present in the bioliquid is increased relative to the same type of waste treated with enzymatic hydrolysis without concurrent fermentation by said one or more species of lactic acid bacteria, acetate-producing bacteria, propionate-producing bacteria, or butyrate-producing bacteria.

2. The method of claim 1, wherein the separating of non-biodegradable solids of the waste is achieved using at least two separation operations sufficient to provide said bioliquid having at least 0.10 kg volatile solids per kg of processed waste.

3. The method of claim 1, wherein inoculation is provided by recycling wash waters or process solutions used to recover residual organic material from non-degradable solids.

4. The method of claim 1, wherein said waste comprises a plastic foil and/or metal foil and wherein said non-biodegradable solids comprise said plastic foil and/or metal foil.

5. The method of claim 1, wherein the waste comprises a non-water content of between 10 and 45% by weight.

6. The method of claim 1, wherein inoculation is performed before or concurrently with the addition of enzymatic activities or with the addition of microorganisms that exhibit extra-cellular cellulase activity.

7. The method of claim 1, wherein cellulase activity is added (i) by inoculation with a selected microorganism that exhibits extra-cellular cellulase activity and/or (ii) as an isolated cellulase preparation.

8. The method of claim 1, wherein microbial fermentation is accomplished by inoculation using one or more species of lactic acid bacteria.

9. The method of claim 1, wherein enzymatic hydrolysis and microbial fermentation are conducted within the temperature range of 45-50° C.

10. The method of claim 1, wherein concurrent enzymatic hydrolysis and microbial fermentation are conducted at a pH of less than 6.0.

11. The method of claim 1, wherein at least 40% by weight of the dissolved volatile solids of the bioliquid comprises lactate, and/or wherein the bioliquid comprises a dissolved methane content at 25 degrees C. of less than 15 mg/L.

12. The method of claim 1, wherein said separation step comprises pressing the liquefied, biodegradable parts of the waste and non-biodegradable solids to produce a MSW bioliquid.

13. A system for the production of a bioliquid, said system comprising:
a) a fermentation vessel at a temperature of 35-75° C. comprising
domestic household waste, wastes from restaurants and food processing facilities, and/or wastes from office buildings,
one or more species of bacteria comprising lactic acid bacteria, acetate-producing bacteria, propionate-producing bacteria, or butyrate-producing bacteria wherein said one or more species of bacteria do not produce ethanol as the primary product of fermentation, and
cellulase enzymes;
b) a means for separating the waste into a solid and liquid fraction; and
c) a bioliquid wherein lactate is present in a higher concentration by weight in the bioliquid when compared to any other single dissolved volatile solid selected from the group consisting of: acetate, butyrate, ethanol, formate, and/or propionate.

14. The system of claim 13, wherein said means for separating the waste into a solid and liquid fraction comprises a ballistic separator.
